(19) United States
(12) Patent Application Publication
(10) Pub. No.: US 2026/0256813 A1
(43) Pub. Date: Sep. 3, 2026

(54) COMPOSITIONS AND METHODS FOR PERIODONTAL CARE

(71) Applicant: ProEncia Biotechnology Inc., Woodbury, MN (US)

(72) Inventors: Tarun Kanti Ghosh, Woodbury, MN (US); Thomas Ellis Hoover, Minneapolis, MN (US); Tapos Kanti Ghosh, Woodbury, MN (US)

(21) Appl. No.: 19/548,447

(22) Filed: Feb. 24, 2026

Related U.S. Application Data
(60) Provisional application No. 63/765,225, filed on Feb. 28, 2025.

(51) Int. Cl. A61K 31/593; A61K 31/192; A61K 31/43; A61K 31/5383; A61K 31/616; A61K 38/12; A61P 1/02

(52) U.S. Cl. CPC A61K 31/593; A61K 31/192; A61K 31/43; A61K 31/5383; A61K 31/616; A61K 38/12; A61P 1/02

(57) ABSTRACT

Compositions for maintaining or improving oral health, and preventing or treating periodontal disease in a subject, and methods for using the same are provided. The composition contains cholecalciferol, at least one reversible cyclooxygenase (COX) inhibitor, and at least one irreversible COX inhibitor. The composition may also contain at least one TLR inhibitor and at least one broad spectrum antibiotic.

### CROSS-REFERENCE TO RELATED APPLICATION

This application claims the benefit of U.S. Provisional Patent Application No. 63/765,225, the entire contents of which are hereby incorporated by reference.

### FIELD OF THE DISCLOSURE

The present disclosure describes compositions and methods for promoting oral health and/or treating periodontal disease.

### BACKGROUND

Periodontal care is required to maintain oral hygiene, periodontium longevity, and oral biome homeostasis. The periodontium is the specialized tissues that surround and support teeth, holding them firmly in the jawbone, and has four main parts: the gingiva (gums), cementum (covering the tooth root), alveolar bone (the jawbone socket), and the periodontal ligament (PDL), which connects the tooth root to the bone, allowing for function and cushioning. Periodontal disease (gingivitis and periodontitis) in humans is caused by oral microbial infection which leads to inflammation, adverse immune responses, progressive tissue damage, and potentially subsequent blood-borne systemic diseases.

Currently, oral periodontal care treatment protocols are dependent on the severity of the disease and include the following: (1) conventional care (brushing, flossing, mouth wash); (2) dental scaling (mechanical debridement procedure to achieve removal of bacterial load, hard plaque and tarter deposits from teeth and root below gumline); (3) dental root planning (a more invasive non-surgical mechanical debridement procedure to achieve deep cleaning, smoothening of root surfaces below detached gum line forming deep gum pockets harboring gum diseases); (4) surgical treatments (including invasive surgical interventions like periodontal pocket reduction or flap surgery, soft tissue graft, bone graft, LANAP (Laser-Assisted New Attachment Procedure); (5) antibiotics treatment; and (6) NSAID (non-steroidal anti-inflammatory drug) treatment (used to control inflammatory symptoms like pain, swelling, tissue damage common to all stages of periodontal disease).

Periodontal pathogens may be aggressive and reside in periodontal pockets which often are out of reach to debridement procedures. Diverse antibiotics are used as an adjunct therapy to debridement, and the application involves oral systemic and intravenous treatment, as well as topical full mouth rinses or gel as a topical gum rub or a palletized insert into the periodontal pocket. However, failed antibiotic treatment is a major danger as it often causes relapse and aggressive progression of periodontal diseases and autoimmunity. This may be caused by incorrect time and concentration, flawed antibiotic delivery to the infection site, failure to eradicate the source of infection, wrong choice of antibiotic, antibiotic-resistant microorganisms, inefficient tissue damage repair, impaired or dysregulated or compromised host immune defenses, and disease transformation to a chronic inflammatory periodontal autoimmune disease (CIPAD).

Thus, there remains a need for a safe, therapeutically effective, and easily deliverable target penetrating, non-invasive, non-toxic oral care composition that prevents and protects from periodontal diseases instigation in disease free individuals or for treating the disease in disease bearing individuals.

It is against this background that the present disclosure is made.

### SUMMARY

A variety of additional inventive aspects will be set forth in the description that follows. The inventive aspects can relate to individual features and to combinations of features. It is to be understood that both the forgoing general description and the following detailed description are exemplary and explanatory only and are not restrictive of the broad inventive concepts upon which the embodiments disclosed herein are based.

Disclosed herein is a composition for maintaining or improving oral health, comprising about 0.001 wt. % to about 0.5 wt. %, of cholecalciferol, based on a total weight of the composition, about 0.01 wt. % to about 15.0 wt. %, of at least one reversible cyclooxygenase COX-2 inhibitor with activity for both bis-oxygenase and peroxidase catalytic arms of COX-2 enzyme, based on a total weight of the composition, and about 0.01 wt. % to about 2.0 wt. %, of at least one irreversible COX-2 inhibitor with selectivity for bis-oxygenase but not COX-2 peroxidase catalytic activity, based on a total weight of the composition. The composition may also comprise at least one of a TLR (Toll-like-receptor) inhibitor as selective as a neutralizer of bacterial endotoxin activity and a broad spectrum antibiotic.

Also disclosed herein is a method of maintaining or improving oral health in a subject, comprising administering an effective amount of a composition described herein to a subject in need thereof, wherein the administering maintains or improves at least one oral health parameter selected from the group consisting of halitosis, tooth sensitivity, gum sensitivity, gum swelling, gum pain and inflammation, gum bleeding, plaque and tarter buildup, and combinations thereof.

Also disclosed herein is a method of preventing or treating periodontal disease in a subject, comprising administering an effective amount of a composition described herein to a subject in need thereof.

### BRIEF DESCRIPTION OF THE DRAWINGS

The accompanying drawings, which are incorporated in and constitute a part of the description, illustrate several aspects of the present disclosure. A brief description of the drawings is as follows:

FIG. 1 depicts a diagram of periodontal cell cultures following pro-inflammatory stimulation before and after treatment with a composition of Example 1.

FIG. 2 is a graphical representation of prostaglandin E2 (PGE2) levels in periodontal cell cultures following pro-inflammatory stimulation before and after treatment with a composition of Example 1.

FIG. 3 is a graphical representation of cytokine levels in periodontal cell cultures following pro-inflammatory stimulation before and after treatment with a composition of Example 1.

FIG. 4 is a graphical representation of specialized pro-resolving mediators (SPM) release levels from periodontal cell in cultures following pro-inflammatory stimulation with or without treatment of a composition of Example 1.

### DETAILED DESCRIPTION

### Definitions

As used herein, weight percent (wt. %), percent by weight, % by weight, and the like are synonyms that refer to the concentration of a substance as the weight of that substance divided by the total weight of the composition and multiplied by 100.

As used herein, weight by volume percent (w/v %), and the like refer to the concentration of a substance as the weight of that substance divided by the total volume of the solution and multiplied by 100. The w/v % may refer to a weight of the substance in grams in 100 mL of the solution. In some examples, when a composition of the present disclosure is formulated as a liquid, such as but not limited to a suspension, a solution, an emulsion, or an emulsified suspension, the components of the composition are referred to as w/v %. One of ordinary skill in the art would recognize how to convert wt. % to w/v % by multiplying the wt. % by the density of the liquid formulation.

As used herein, the term “about” modifying the quantity of an ingredient in the compositions of the invention or employed in the methods of the invention refers to variation in the numerical quantity that can occur, for example, through typical measuring and liquid handling procedures used for making use solutions in the real world; through inadvertent error in these procedures; through differences in the manufacture, source, or purity of the ingredients employed to make the compositions or carry out the methods; and the like. The term about also encompasses amounts that differ due to different equilibrium conditions for a composition resulting from a particular initial mixture. Whether or not modified by the term “about,” the claims include equivalents to the quantities. The term “about” typically allows for a variation within ±5% of the stated percent value. For example, if “about 10%” is used, the term “about” typically allows for a variation of about 5% of the stated percent value, 10%. Thus, “about 10%” may cover a variation of ±0.5%.

As used herein, the terms “substantially free,” “substantially free of,” and “free of” of a particular substance means that the compositions of the instant specification contain less than 0.5 wt. % of the recited substance. When referring to “substantially free,” “substantially free of,” and “free of” it is intended that the substance is not intentionally added to the compositions. The term “essentially free” of a particular substance means that the compositions of the instant specification contain less than 0.1 wt. % of the recited substance. When referring to “essentially free” it is intended that the substance is not intentionally added to the compositions. The term “essentially completely free” of a particular substance means that the compositions of the instant specification contain less than 0.01 wt. % of the recited substance. When referring to “essentially completely free” it is intended that the substance is not intentionally added to the compositions. The term “completely free” of a particular substance means that the compositions of the instant specification contain less than 0.001 wt. % of the recited substance. When referring to “completely free” it is intended that the substance is not intentionally added to the compositions. Use of the term “completely free” allows for trace amounts of that substance to be included in compositions because they are present in another substance in the composition. However, it is recognized that only trace or de minimus amounts of a substance will be allowed when the composition is said to be “completely free” of that substance.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

In the interest of brevity and conciseness, any ranges of values set forth in this specification contemplate all values within the range and are to be construed as support for claims reciting any sub-ranges having endpoints which are real number values within the specified range in question. By way of a hypothetical illustrative example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5.

As used herein, the term “consisting essentially of” refers to the listed ingredients and does not include additional ingredients that, if present, would affect the treatment ability of the compositions disclosed herein. As used herein the term “consisting essentially of” in reference to a method refers to the listed steps and does not include additional steps (or ingredients if a composition is included in the method) that, if present, would affect the method.

As used herein, the term “consisting of” refers to the listed ingredients and does not include additional ingredients. In some examples, impurities may be present in addition to the listed ingredients, however, the impurities are not present in an amount greater than 0.01 wt. %, based on a total weight of the composition and are not intentionally added. The term “consisting of” in reference to a method refers to the listed steps and does not include additional steps (or ingredients if a composition is included in the method).

As used herein, each compound refers to the compound in free acid or base form or any pharmaceutically acceptable salt, ester, hydrate, solvate, polymorph, deuterated derivative, N-oxide, clathrate, prodrug, or stereoisomer thereof, or a combination thereof. For example, in the compositions of the present disclosure, certain compounds may be present, such as but not limited to, ibuprofen. One of ordinary skill in the art would understand that ibuprofen is commonly found as a sodium salt, however, in the compositions of the present disclosure, each component listed may refer to any salts thereof. For example, ibuprofen may refer to free acid ibuprofen, a sodium salt of ibuprofen, a lysine salt of ibuprofen, or any salt thereof, unless otherwise specified. Salts can denote acidic and/or basic salts formed with inorganic and/or organic acids and bases. When a compound contains both basic and acidic moieties, zwitterions (“inner salts”) may form. Pharmaceutically acceptable salts include those generally acceptable in pharmaceutical sciences for administration to subjects, including humans and animals; these salts can be non-toxic and physiologically acceptable. Compounds containing basic moieties may form salts with organic and inorganic acids. Examples include, but are not limited to, acetates, adipates, alginates, ascorbates, aspartates, benzoates, benzenesulfonates, bisulfates, borates, butyrates, citrates, camphorates, camphorsulfonates, cyclopentanepropionates, digluconates, dodecyl sulfates, ethanesulfonates, fumarates, glucoheptanoates, glycerophosphates, hemisulfates, heptanoates, hexanoates, hydrochlorides, hydrobromides, hydroiodides, lactates, maleates, methanesulfonates, nicotinates, nitrates, oxalates, pectinates, persulfates, phosphates, picrates, pivalates, propionates, salicylates, succinates, sulfates, sulfonates, tartrates, thiocyanates, toluenesulfonates, and undecanoates. Compounds containing acidic moieties may form salts with organic and inorganic bases. Examples include, but are not limited to, ammonium salts, alkali metal salts (sodium, lithium, potassium), alkaline earth metal salts (calcium, magnesium), salts with organic bases (benzathines, dicyclohexylamines, hydrabamines, N-methyl-D-glucamines, N-methyl-D-glucamides, t-butyl amines and the like), and salts with amino acids such as arginine and lysine. Basic nitrogen-containing groups may be quaternized with agents such as lower alkyl halides, dialkyl sulfates, long chain halides, and aralkyl halides. This also applies to esters, hydrates, solvates, polymorphs, deuterated derivatives, N-oxides, clathrates, prodrugs, and stereoisomers of the compounds, where the listed compound may be any ester, hydrate, solvate, polymorph, deuterated derivative, N-oxide, clathrate, prodrug, or stereoisomer of said compound.

As used herein, unless otherwise indicated, all amounts and weight percentages of compounds described herein are expressed on a free acid or free base equivalent basis, irrespective of whether the compound is present as a salt, hydrate, solvate, polymorph, or other solid-state form.

As used herein, the term “subject” includes, but is not limited to, humans and non-human vertebrates such as wild, domestic, and farm animals. In some examples, the subject described herein is an animal. In some examples, the subject is a mammal. In some examples, the subject is a human. In some examples, the subject is a non-human animal. In some examples, the subject is a non-human mammal. In some examples, the subject is a domesticated animal, such as a dog, cat, cow, pig, horse, sheep, or goat. In some examples, the subject is a companion animal such as a dog or cat. In some examples, the subject is a livestock animal such as a cow, pig, horse, sheep, or goat. In some examples, the subject is a zoo animal. In some examples, the subject is a research animal such as a rodent, dog, or non-human primate. In some examples, the subject is a non-human transgenic animal such as a transgenic mouse or transgenic pig.

As used herein, “active ingredient(s)”, “active compound(s),” and the like are a component of a composition of the present disclosure that is intended to provide a pharmacological, therapeutic, prophylactic, or biological effect, as distinguished from excipients, carriers, or other inactive components.

As used herein, “administering” when used in conjunction with an active pharmaceutical ingredient, can include, but is not limited to, providing the active pharmaceutical ingredient into or onto the target tissue; providing the active pharmaceutical ingredient systemically to a patient by, e.g., injection, topical administration, orally, or by either method in combination with other known techniques.

As used herein, the terms “treating” or “treatment” refer to and include treatment of a disease-state in a subject, for example in a human or animal, and include: (a) inhibiting the disease-state, i.e., arresting its development; (b) relieving the disease-state, i.e., causing regression of the disease state; and/or (c) preventing the disease-state from occurring in a subject. In some examples, treatment includes relieving or one or more symptoms of a disease-state in a subject. In some examples, treatment may be administered after one or more symptoms have developed. In other examples, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the terms “preventing” or “prevention” refer to and include preventive treatment (i.e., prophylaxis and/or risk reduction) of a subclinical disease-state in a subject, for example in a human or animal, aimed at reducing the probability of the occurrence of a clinical disease-state. Subjects may be selected for preventative therapy based on factors that are known to increase risk of suffering a clinical disease state compared to the general population. “Prophylaxis” therapies can be divided into (a) primary prevention, and (b) secondary prevention. Primary prevention is defined as treatment in a subject that has not yet presented with a clinical disease state, whereas secondary prevention is defined as preventing a second occurrence of the same or similar clinical disease state.

As used herein, the term “effective amount” refers to an amount of a substance (e.g., a therapeutic agent, composition, and/or formulation), that elicits a desired biological response. In some examples, an effective amount of a substance is an amount that is sufficient, when administered as part of a dosing regimen to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a provided compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some examples, an effective amount is at least a minimal amount of a provided compound, or composition containing a provided compound, which is sufficient for treating one or more symptoms of a periodontal disease. When applied to a combination, the term refers to combined amounts of the active ingredients that result in the preventive or therapeutic effect, whether administered in combination, serially, or simultaneously.

As used herein, the term “maintain’ or “maintaining” is used to convey that the composition of the prevent disclosure aid in preserving or sustaining the current condition or functional integrity of the tissue, for example in the oral cavity of the subject.

As used herein, the term “improve” or “improving” is used to convey that the compositions of the present disclosure change either the appearance, form, characteristics and/or the physical attributes of the tissue to which it is being provided, applied or administered. In some examples, improving oral health includes treating, preventing, reducing, controlling, stabilizing, or delaying the onset or progression of one or more oral health conditions or symptoms.

As used herein, the term “oral health” refers to the condition and functional integrity of the oral cavity of a subject, including the teeth, gums, periodontal tissues, oral mucosa, and supporting structures, and encompasses the absence, reduction, or control of disease, inflammation, infection, discomfort, or functional impairment affecting the oral cavity.

As used herein, the term “periodontal disease”, refers to a group of conditions that affect the periodontium, the supporting structures of the teeth, including the gingiva, alveolar bone, cementum, and periodontal ligament.

### Compositions

The compositions of the present disclosure improve oral health, such as but not limited to halitosis, tooth sensitivity, gum sensitivity, gum swelling, gum pain and inflammation, gum bleeding, plaque and tarter buildup, and combinations thereof, described in further detail below. The compositions of the present disclosure may also prevent or treat periodontal diseases, described in further detail below.

In some examples, the compositions of the present disclosure may comprise at least one vitamin. In some examples, the at least one vitamin is selected from group consisting of Vitamin A, Vitamin E, Vitamin D, Vitamin K, and Vitamin C. In some examples, the at least one vitamin is selected from group consisting of Vitamin D2 (ergocalciferol) and Vitamin D3 (cholecalciferol). In some examples, the compositions of the present disclosure may comprise cholecalciferol. Cholecalciferol (Vitamin D3) is an innate and adaptive immune defense system protective, pathogen invasion and infectibility deterrent, and secosteroid pro-hormone.

In some examples, the vitamin is included in the composition in an amount of about 0.001 wt. % to about 1.0 wt. %, based on a total weight of the composition. In some examples, the vitamin is included in the composition in an amount of about 0.001 wt. % to about 1.0 wt. %, about 0.005 wt. % to about 0.5 wt. %, about 0.01 wt. % to about 0.1 wt. %, or about 0.05 wt. %, based on a total weight of the composition.

In some examples, the compositions of the present disclosure may comprise at least one enzyme inhibitor. In some examples, the at least one enzyme inhibitor may be, but is not limited to, a cyclooxygenase (COX) inhibitor and a 5-lipoxygenase (5LO) inhibitor. In some examples the 5LO inhibitor includes compounds capable of inhibiting leukotriene biosynthesis, including, but not limited to, pyrazole-containing compounds, sulfonamide derivatives, benzoxazole derivatives, indole derivatives, and other small-molecule scaffolds exhibiting 5-lipoxygenase inhibitory activity. In some examples the 5LO inhibitor is zileuton, licofelone, tepoxalin, and other small-molecule compounds such as pyrazole, sulfonamide, benzoxazole, indole, quinoline, and hydroxyurea derivatives that inhibit leukotriene biosynthesis.

In some examples, the compositions of the present disclosure may comprise at least one COX inhibitor. In some examples, a COX inhibitor is a compound that reduces or blocks the activity of cyclooxygenase enzymes (one or more of COX-1, COX-2, and COX-3), thereby decreasing the production of prostaglandins involved in inflammation, pain, or other physiological processes. In some examples, the COX inhibitor is an inhibitor of COX-1. In some examples, the COX inhibitor is an inhibitor of COX-2. In some examples, the COX inhibitor is an inhibitor of COX-1 and COX-2. In some examples, the compositions of the present disclosure may comprise at least one reversible COX inhibitor. In some examples, the COX inhibitor being reversible means that the inhibitor binds to COX in a non-permanent manner, such that the inhibitory effect can be diminished, undone, or displaced upon removal of the inhibitor or under physiological conditions. In some examples, the compositions of the present disclosure may comprise at least one nonsteroidal anti-inflammatory drug (NSAID).

In some examples, the reversible COX inhibitor is any reversible COX inhibitor known in the art. In some examples, the reversible COX inhibitor is selected from the group consisting of ibuprofen, naproxen, diclofenac, indomethacin, ketoprofen, flurbiprofen, piroxicam, meloxicam, sulindac, tolmetin, etodolac, nabumetone, celecoxib, and etoricoxib. In some examples, the reversible COX inhibitor is ibuprofen.

In some examples, the reversible COX inhibitor is included in the composition in an amount of about 0.01 wt. % to about 15.0 wt. %, based on a total weight of the composition. In some examples, the reversible COX inhibitor is included in the composition in an amount of about 0.01 wt. % to about 15.0 wt. %, about 0.5 wt. % to about 14.5 wt. %, about 1.0 wt. % to about 14.0 wt. %, about 1.5 wt. % to about 13.5 wt. %, about 2.0 wt. % to about 13.0 wt. %, about 2.5 wt. % to about 12.5 wt. %, about 3.0 wt. % to about 12.0 wt. %, about 3.5 wt. % to about 11.5 wt. %, about 4.0 wt. % to about 11.0 wt. %, about 4.5 wt. % to about 10.5 wt. %, about 5.0 wt. % to about 10.0 wt. %, about 5.5 wt. % to about 9.5 wt. %, about 6.0 wt. % to about 9.0 wt. %, about 6.5 wt. % to about 8.5 wt. %, about 7.0 wt. % to about 8.0 wt. %, or about 7.5 wt. %, based on a total weight of the composition.

In some examples, the compositions of the present disclosure may comprise at least one irreversible COX inhibitor. In some examples, the COX inhibitor being irreversible means that the inhibitor binds to COX in a permanent or covalent manner, such that the inhibitory effect cannot be readily reversed or displaced under physiological conditions, leading to lasting inactivation of COX.

In some examples, the irreversible COX inhibitor is any irreversible COX inhibitor known in the art. In some example, the irreversible COX inhibitor is selective for COX bis-oxygenase catalytic activity but not COX peroxidase enzyme catalytic activity. In some examples, the irreversible COX inhibitor is a COX bis-oxygenase catalytic activity selective inhibitor aspirin.

In some examples, the irreversible COX inhibitor is included in the composition in an amount of about 0.001 wt. % to about 15.0 wt. %, based on a total weight of the composition. In some examples, the irreversible COX inhibitor is included in the composition in an amount of about 0.001 wt. % to about 15.0 wt. %, about 0.005 wt. % to about 15.0 wt. %, about 0.01 wt. % to about 15.0 wt. %, about 0.5 wt. % to about 14.5 wt. %, about 1.0 wt. % to about 14.0 wt. %, about 1.5 wt. % to about 13.5 wt. %, about 2.0 wt. % to about 13.0 wt. %, about 2.5 wt. % to about 12.5 wt. %, about 3.0 wt. % to about 12.0 wt. %, about 3.5 wt. % to about 11.5 wt. %, about 4.0 wt. % to about 11.0 wt. %, about 4.5 wt. % to about 10.5 wt. %, about 5.0 wt. % to about 10.0 wt. %, about 5.5 wt. % to about 9.5 wt. %, about 6.0 wt. % to about 9.0 wt. %, about 6.5 wt. % to about 8.5 wt. %, about 7.0 wt. % to about 8.0 wt. %, or about 7.5 wt. %, based on a total weight of the composition.

In some examples, a composition of the present disclosure comprises a ratio by weight of the at least one reversible COX inhibitor to the at least one irreversible COX inhibitor in the composition from about 10:1 to about 5:1. In some examples, a composition of the present disclosure comprises a ratio by weight of the at least one reversible COX inhibitor to the at least one irreversible COX inhibitor in the composition from about 10:1 to about 5:1, about 9:1 to about 6:1, or about 8:1 to about 7:1.

In some examples, a first composition of the present disclosure comprises the at least one vitamin in an amount of about 0.001 wt. % to about 1.0 wt. %, about 0.005 wt. % to about 0.5 wt. %, about 0.01 wt. % to about 0.1 wt. %, or about 0.05 wt. %, based on a total weight of the composition; the at least one reversible COX inhibitor in an amount of about 0.01 wt. % to about 15.0 wt. %, about 0.5 wt. % to about 14.5 wt. %, about 1.0 wt. % to about 14.0 wt. %, about 1.5 wt. % to about 13.5 wt. %, about 2.0 wt. % to about 13.0 wt. %, about 2.5 wt. % to about 12.5 wt. %, about 3.0 wt. % to about 12.0 wt. %, about 3.5 wt. % to about 11.5 wt. %, about 4.0 wt. % to about 11.0 wt. %, about 4.5 wt. % to about 10.5 wt. %, about 5.0 wt. % to about 10.0 wt. %, about 5.5 wt. % to about 9.5 wt. %, about 6.0 wt. % to about 9.0 wt. %, about 6.5 wt. % to about 8.5 wt. %, about 7.0 wt. % to about 8.0 wt. %, or about 7.5 wt. %, based on a total weight of the composition; and the at least one irreversible COX inhibitor in an amount of about 0.01 wt. % to about 15.0 wt. %, about 0.5 wt. % to about 14.5 wt. %, about 1.0 wt. % to about 14.0 wt. %, about 1.5 wt. % to about 13.5 wt. %, about 2.0 wt. % to about 13.0 wt. %, about 2.5 wt. % to about 12.5 wt. %, about 3.0 wt. % to about 12.0 wt. %, about 3.5 wt. % to about 11.5 wt. %, about 4.0 wt. % to about 11.0 wt. %, about 4.5 wt. % to about 10.5 wt. %, about 5.0 wt. % to about 10.0 wt. %, about 5.5 wt. % to about 9.5 wt. %, about 6.0 wt. % to about 9.0 wt. %, about 6.5 wt. % to about 8.5 wt. %, about 7.0 wt. % to about 8.0 wt. %, or about 7.5 wt. %, based on a total weight of the composition.

In some examples, a first composition of the present disclosure comprises cholecalciferol in an amount of about 0.001 wt. % to about 1.0 wt. %, about 0.005 wt. % to about 0.5 wt. %, about 0.01 wt. % to about 0.1 wt. %, or about 0.05 wt. %, based on a total weight of the composition; ibuprofen in an amount of about 0.01 wt. % to about 15.0 wt. %, about 0.5 wt. % to about 14.5 wt. %, about 1.0 wt. % to about 14.0 wt. %, about 1.5 wt. % to about 13.5 wt. %, about 2.0 wt. % to about 13.0 wt. %, about 2.5 wt. % to about 12.5 wt. %, about 3.0 wt. % to about 12.0 wt. %, about 3.5 wt. % to about 11.5 wt. %, about 4.0 wt. % to about 11.0 wt. %, about 4.5 wt. % to about 10.5 wt. %, about 5.0 wt. % to about 10.0 wt. %, about 5.5 wt. % to about 9.5 wt. %, about 6.0 wt. % to about 9.0 wt. %, about 6.5 wt. % to about 8.5 wt. %, about 7.0 wt. % to about 8.0 wt. %, or about 7.5 wt. %, based on a total weight of the composition; and aspirin in an amount of about 0.01 wt. % to about 15.0 wt. %, about 0.5 wt. % to about 14.5 wt. %, about 1.0 wt. % to about 14.0 wt. %, about 1.5 wt. % to about 13.5 wt. %, about 2.0 wt. % to about 13.0 wt. %, about 2.5 wt. % to about 12.5 wt. %, about 3.0 wt. % to about 12.0 wt. %, about 3.5 wt. % to about 11.5 wt. %, about 4.0 wt. % to about 11.0 wt. %, about 4.5 wt. % to about 10.5 wt. %, about 5.0 wt. % to about 10.0 wt. %, about 5.5 wt. % to about 9.5 wt. %, about 6.0 wt. % to about 9.0 wt. %, about 6.5 wt. % to about 8.5 wt. %, about 7.0 wt. % to about 8.0 wt. %, or about 7.5 wt. %, based on a total weight of the composition.

In some examples, the first composition may further comprise at least one excipient selected from the group consisting of a carrier, a surfactant, a emollient, a thickening agent, a buffering agent, a sweetening agent, a flavoring agent, a coloring agent, an antioxidant, a chelating agent, a preservative, a gelling agent, and combinations thereof, as described below.

In some examples, the first composition may be used to improve or maintain an oral health of a subject such as but not limited to maintaining or improving at least one oral health parameter selected from the group consisting of halitosis, tooth sensitivity, gum sensitivity, gum swelling, gum pain and inflammation, gum bleeding, plaque and tarter buildup, and combinations thereof. Without wishing to be bound by theory, the individual components included in the first composition described herein may yield limited benefit, while the combination work in synergy and offer a desired therapeutic benefit with limitation of a toxicity profile of the individual components. The components may act in synergy as tissue protective, tissue damage repair, and bone remodeling activator agents to safeguard and improve condition of the periodontal immune system and provide clinical improvement of periodontal parameters such as plaque index, gingival index, probing depth, clinical attachment loss, bone loss, and sensitivity from gingival recession.

In some examples, the compositions of the present disclosure may comprise at least one Toll-like receptor (TLR) inhibitor. TLRs are a family of pattern recognition receptors (PRRs) of the innate and adaptive immune system that recognize pathogen-associated molecular patterns (PAMPs) and damage-associated molecular patterns (DAMPs) and initiate intracellular signaling pathways that regulate inflammatory and immune responses. The TLRs may recognize, for example, pro-inflammatory bacterial and viral toxins like endotoxin lipopolysaccharides (LPS, TLR4-agonist/ligand), zymosan (TLR2-agonist/ligand), and/or adaptive immune system activator like cytosine-phosphate-guanine oligodeoxynucleotides (CPG, TLR9-agonist/ligand). A TLR inhibitor refers to any compound that reduces, blocks, or modulates the activity of one or more TLRs, for example by affecting TLR-mediated signaling induced by PAMPs or DAMPs, thereby diminishing or regulating the downstream signaling pathways involved in inflammation, immune response, or other TLR-mediated physiological processes.

TLR inhibitors may include small molecules, peptides, antibodies, aptamers, nucleic acids, or other agents that inhibit one or more TLRs. In some examples, the TLR inhibitor inhibits at least one selected from the group consisting of TLR1, TLR2, TLR3, TLR4, TLR5, TLR6, TLR7, TLR8, TLR9, and TLR10. In some examples, the TLR inhibitor inhibits at least one selected from the group consisting of TLR2, TLR4, and TLR9. In some examples, the TLR inhibitor inhibits TLR4 and/or TLR2.

In some examples, the TLR inhibitor is any TLR inhibitor known in the art. In some examples, the TLR inhibitor is selected from the group consisting of TLR1/TLR2 inhibitors: OPN-305, CU-CPT22, Pam3CSK4 antagonists, lipoprotein-derived antagonists; TLR2 inhibitors: OPN-305, CU-CPT22, sparstolonin B, OxPAPC, T2.5 peptide; TLR3 inhibitors: poly(I: C) antagonists, phenylmethimazole, resveratrol; TLR4 inhibitors: eritoran (E5564), TAK-242 (resatorvid), polymyxin B, polymyxin E (colistin), lipid A analog antagonists, OxPAPC; TLR7 inhibitors: IRS-661, chloroquine, hydroxychloroquine; TLR8 inhibitors: CU-CPT9a, chloroquine; and TLR9 inhibitors: ODN-2088, ODN-20958, chloroquine, inhibitory CpG oligonucleotides.

In some examples, the TLR inhibitor is a TLR4 inhibitor selected from the group consisting of resatorvid (TAK-242), eritoran, polymyxin B and polymyxin E. In some examples, the TLR inhibitor is polymyxin B. In some examples, the TLR binding inhibitor is polymyxin B, polymyxin E, or a combination thereof.

In some examples, the TLR inhibitor is included in the composition in an amount of about 0.001 wt. % to about 0.5 wt. %, based on a total weight of the composition. In some examples, the TLR inhibitor is included in the composition in an amount of about 0.001 wt. % to about 0.5 wt. %, about 0.005 wt. % to about 0.1 wt. %, or about 0.01 wt. % to about 0.05 wt. %, based on a total weight of the composition.

In some examples, a second composition of the present disclosure comprises at least one vitamin in an amount of about 0.001 wt. % to about 1.0 wt. %, about 0.005 wt. % to about 0.5 wt. %, about 0.01 wt. % to about 0.1 wt. %, or about 0.05 wt. %, based on a total weight of the composition; the at least one reversible COX inhibitor in an amount of about 0.01 wt. % to about 15.0 wt. %, about 0.5 wt. % to about 14.5 wt. %, about 1.0 wt. % to about 14.0 wt. %, about 1.5 wt. % to about 13.5 wt. %, about 2.0 wt. % to about 13.0 wt. %, about 2.5 wt. % to about 12.5 wt. %, about 3.0 wt. % to about 12.0 wt. %, about 3.5 wt. % to about 11.5 wt. %, about 4.0 wt. % to about 11.0 wt. %, about 4.5 wt. % to about 10.5 wt. %, about 5.0 wt. % to about 10.0 wt. %, about 5.5 wt. % to about 9.5 wt. %, about 6.0 wt. % to about 9.0 wt. %, about 6.5 wt. % to about 8.5 wt. %, about 7.0 wt. % to about 8.0 wt. %, or about 7.5 wt. %, based on a total weight of the composition; the at least one irreversible COX inhibitor in an amount of about 0.01 wt. % to about 15.0 wt. %, about 0.5 wt. % to about 14.5 wt. %, about 1.0 wt. % to about 14.0 wt. %, about 1.5 wt. % to about 13.5 wt. %, about 2.0 wt. % to about 13.0 wt. %, about 2.5 wt. % to about 12.5 wt. %, about 3.0 wt. % to about 12.0 wt. %, about 3.5 wt. % to about 11.5 wt. %, about 4.0 wt. % to about 11.0 wt. %, about 4.5 wt. % to about 10.5 wt. %, about 5.0 wt. % to about 10.0 wt. %, about 5.5 wt. % to about 9.5 wt. %, about 6.0 wt. % to about 9.0 wt. %, about 6.5 wt. % to about 8.5 wt. %, about 7.0 wt. % to about 8.0 wt. %, or about 7.5 wt. %, based on a total weight of the composition; and the at least one TLR inhibitor in an amount of about 0.001 wt. % to about 0.5 wt. %, about 0.005 wt. % to about 0.1 wt. %, or about 0.01 wt. % to about 0.05 wt. %, based on a total weight of the composition.

In some examples, a second composition of the present disclosure comprises cholecalciferol in an amount of about 0.001 wt. % to about 1.0 wt. %, about 0.005 wt. % to about 0.5 wt. %, about 0.01 wt. % to about 0.1 wt. %, or about 0.05 wt. %, based on a total weight of the composition; ibuprofen in an amount of about 0.01 wt. % to about 15.0 wt. %, about 0.5 wt. % to about 14.5 wt. %, about 1.0 wt. % to about 14.0 wt. %, about 1.5 wt. % to about 13.5 wt. %, about 2.0 wt. % to about 13.0 wt. %, about 2.5 wt. % to about 12.5 wt. %, about 3.0 wt. % to about 12.0 wt. %, about 3.5 wt. % to about 11.5 wt. %, about 4.0 wt. % to about 11.0 wt. %, about 4.5 wt. % to about 10.5 wt. %, about 5.0 wt. % to about 10.0 wt. %, about 5.5 wt. % to about 9.5 wt. %, about 6.0 wt. % to about 9.0 wt. %, about 6.5 wt. % to about 8.5 wt. %, about 7.0 wt. % to about 8.0 wt. %, or about 7.5 wt. %, based on a total weight of the composition; aspirin in an amount of about 0.01 wt. % to about 15.0 wt. %, about 0.5 wt. % to about 14.5 wt. %, about 1.0 wt. % to about 14.0 wt. %, about 1.5 wt. % to about 13.5 wt. %, about 2.0 wt. % to about 13.0 wt. %, about 2.5 wt. % to about 12.5 wt. %, about 3.0 wt. % to about 12.0 wt. %, about 3.5 wt. % to about 11.5 wt. %, about 4.0 wt. % to about 11.0 wt. %, about 4.5 wt. % to about 10.5 wt. %, about 5.0 wt. % to about 10.0 wt. %, about 5.5 wt. % to about 9.5 wt. %, about 6.0 wt. % to about 9.0 wt. %, about 6.5 wt. % to about 8.5 wt. %, about 7.0 wt. % to about 8.0 wt. %, or about 7.5 wt. %, based on a total weight of the composition; and polymyxin B in an amount of about 0.001 wt. % to about 0.5 wt. %, about 0.005 wt. % to about 0.1 wt. %, or about 0.01 wt. % to about 0.05 wt. %, based on a total weight of the composition.

In some examples, the second composition may further comprise at least one excipient selected from the group consisting of a carrier, a surfactant, a emollient, a thickening agent, a buffering agent, a sweetening agent, a flavoring agent, a coloring agent, an antioxidant, a chelating agent, a preservative, a gelling agent, and combinations thereof, as described below.

In some examples, the second composition may be used to treat periodontal disease and/or bacterial infection. Without wishing to be bound by theory, the second composition described herein may serve, in addition to the benefits described above, to synergistically support selective binding neutralization and inactivating bacterial endotoxins LPS and necrotic host tissue derived toxins, inhibit adverse pro-inflammatory activity, deter bacterial antibiotic resistance (MDR) in pathogenic infection, and help restore system order and homeostasis against dysregulated inflammatory immune responses in both acute and chronic periodontal diseases associated stressed conditions.

In some examples, the compositions of the present disclosure may comprise at least one antibiotic. In some examples, the compositions of the present disclosure may comprise at least one antibiotic which targets gram-positive and/or gram-negative bacteria. In some examples, the compositions of the present disclosure may comprise at least one broad-spectrum antibiotic. Broad-spectrum antibiotic means an antimicrobial agent that is effective against a wide range of bacterial species, including both gram-positive and gram-negative bacteria, and is not limited to activity against a single genus or narrow group of organisms. In some examples, the antibiotic targets bacterial membrane synthesis. In some examples, the antibiotic targets bacterial DNA replication. In some examples, the antibiotic targets bacterial membrane synthesis and/or bacterial DNA replication. In some examples, a combination of antibiotics targets both bacterial membrane synthesis and bacterial DNA replication.

In some examples, the compositions of the present disclosure may comprise at least one broad-spectrum antibiotic from a class selected from the group consisting of β-lactams, tetracyclines, fluoroquinolones, sulfonamides, chloramphenicol, macrolides, nitroimidazoles, and combinations thereof. In some examples, the compositions of the present disclosure may comprise at least one broad-spectrum antibiotic selected from the group consisting of amoxicillin, amoxicillin-clavulanate, ampicillin, imipenem, eropenem, doripenem, ertapenem piperacillin, piperacillin-tazobactam, ticarcillin, cephalosporins, carbapenems, monobactams, tetracycline, doxycycline, minocycline, ciprofloxacin, levofloxacin, moxifloxacin, sulfamethoxazole, sulfisoxazole, sulfadiazine, chloramphenicol, and combinations thereof. In some examples, the compositions of the present disclosure may comprise at least one broad-spectrum antibiotic which is amoxicillin. In some examples, the compositions of the present disclosure may comprise at least one broad-spectrum antibiotic which is amoxicillin-clavulanate. In some examples, the compositions of the present disclosure may comprise at least one broad-spectrum antibiotic which is levofloxacin. In some examples, the compositions of the present disclosure may comprise at least one broad-spectrum antibiotic which is a combination of amoxicillin and levofloxacin. In some examples, the compositions of the present disclosure may comprise at least one broad-spectrum antibiotic which is a combination of amoxicillin-clavulanate and levofloxacin.

In some examples, the composition comprises from about 0.01 wt. % to about 10.0 wt. %, of each of the at least one broad-spectrum antibiotic, based on a total weight of the composition. In other words, if more than one broad-spectrum antibiotic is included in the composition, for example two, there may be about 0.01 wt. % to about 10.0 wt. % of a first antibiotic and about 0.01 wt. % to about 10.0 wt. % of a second antibiotic. In some examples, the composition comprises from about 0.01 wt. % to about 10.0 wt. %, about 0.01 wt. % to about 9.0 wt. %, about 0.01 wt. % to about 8.0 wt. %, about 0.01 wt. % to about 7.0 wt. %, about 0.01 wt. % to about 6.0 wt. %, about 0.01 wt. % to about 5.0 wt. %, 0.05 wt. % to about 4.5 wt. %, 0.1 wt. % to about 4.0 wt. %, 0.5 wt. % to about 3.5 wt. %, 1.0 wt. % to about 3.0 wt. %, 1.5 wt. % to about 2.5 wt. %, or about 2.0 wt. % of each of the at least one broad-spectrum antibiotic, based on a total weight of the composition.

In some examples, a third composition of the present disclosure comprises at least one vitamin in an amount of about 0.001 wt. % to about 1.0 wt. %, about 0.005 wt. % to about 0.5 wt. %, about 0.01 wt. % to about 0.1 wt. %, or about 0.05 wt. %, based on a total weight of the composition; the at least one reversible COX inhibitor in an amount of about 0.01 wt. % to about 15.0 wt. %, about 0.5 wt. % to about 14.5 wt. %, about 1.0 wt. % to about 14.0 wt. %, about 1.5 wt. % to about 13.5 wt. %, about 2.0 wt. % to about 13.0 wt. %, about 2.5 wt. % to about 12.5 wt. %, about 3.0 wt. % to about 12.0 wt. %, about 3.5 wt. % to about 11.5 wt. %, about 4.0 wt. % to about 11.0 wt. %, about 4.5 wt. % to about 10.5 wt. %, about 5.0 wt. % to about 10.0 wt. %, about 5.5 wt. % to about 9.5 wt. %, about 6.0 wt. % to about 9.0 wt. %, about 6.5 wt. % to about 8.5 wt. %, about 7.0 wt. % to about 8.0 wt. %, or about 7.5 wt. %, based on a total weight of the composition; the at least one irreversible COX inhibitor in an amount of about 0.01 wt. % to about 15.0 wt. %, about 0.5 wt. % to about 14.5 wt. %, about 1.0 wt. % to about 14.0 wt. %, about 1.5 wt. % to about 13.5 wt. %, about 2.0 wt. % to about 13.0 wt. %, about 2.5 wt. % to about 12.5 wt. %, about 3.0 wt. % to about 12.0 wt. %, about 3.5 wt. % to about 11.5 wt. %, about 4.0 wt. % to about 11.0 wt. %, about 4.5 wt. % to about 10.5 wt. %, about 5.0 wt. % to about 10.0 wt. %, about 5.5 wt. % to about 9.5 wt. %, about 6.0 wt. % to about 9.0 wt. %, about 6.5 wt. % to about 8.5 wt. %, about 7.0 wt. % to about 8.0 wt. %, or about 7.5 wt. %, based on a total weight of the composition; the at least one TLR inhibitor in an amount of about 0.001 wt. % to about 0.5 wt. %, about 0.005 wt. % to about 0.1 wt. %, or about 0.01 wt. % to about 0.05 wt. %, based on a total weight of the composition; and each of the at least one broad-spectrum antibiotic in an amount of about 0.01 wt. % to about 10.0 wt. %, about 0.01 wt. % to about 9.0 wt. %, about 0.01 wt. % to about 8.0 wt. %, about 0.01 wt. % to about 7.0 wt. %, about 0.01 wt. % to about 6.0 wt. %, about 0.01 wt. % to about 5.0 wt. %, 0.05 wt. % to about 4.5 wt. %, 0.1 wt. % to about 4.0 wt. %, 0.5 wt. % to about 3.5 wt. %, 1.0 wt. % to about 3.0 wt. %, 1.5 wt. % to about 2.5 wt. %, or about 2.0 wt. %, based on a total weight of the composition.

In some examples, a third composition of the present disclosure comprises cholecalciferol in an amount of about 0.001 wt. % to about 1.0 wt. %, about 0.005 wt. % to about 0.5 wt. %, about 0.01 wt. % to about 0.1 wt. %, or about 0.05 wt. %, based on a total weight of the composition; ibuprofen in an amount of about 0.01 wt. % to about 15.0 wt. %, about 0.5 wt. % to about 14.5 wt. %, about 1.0 wt. % to about 14.0 wt. %, about 1.5 wt. % to about 13.5 wt. %, about 2.0 wt. % to about 13.0 wt. %, about 2.5 wt. % to about 12.5 wt. %, about 3.0 wt. % to about 12.0 wt. %, about 3.5 wt. % to about 11.5 wt. %, about 4.0 wt. % to about 11.0 wt. %, about 4.5 wt. % to about 10.5 wt. %, about 5.0 wt. % to about 10.0 wt. %, about 5.5 wt. % to about 9.5 wt. %, about 6.0 wt. % to about 9.0 wt. %, about 6.5 wt. % to about 8.5 wt. %, about 7.0 wt. % to about 8.0 wt. %, or about 7.5 wt. %, based on a total weight of the composition; aspirin in an amount of about 0.01 wt. % to about 15.0 wt. %, about 0.5 wt. % to about 14.5 wt. %, about 1.0 wt. % to about 14.0 wt. %, about 1.5 wt. % to about 13.5 wt. %, about 2.0 wt. % to about 13.0 wt. %, about 2.5 wt. % to about 12.5 wt. %, about 3.0 wt. % to about 12.0 wt. %, about 3.5 wt. % to about 11.5 wt. %, about 4.0 wt. % to about 11.0 wt. %, about 4.5 wt. % to about 10.5 wt. %, about 5.0 wt. % to about 10.0 wt. %, about 5.5 wt. % to about 9.5 wt. %, about 6.0 wt. % to about 9.0 wt. %, about 6.5 wt. % to about 8.5 wt. %, about 7.0 wt. % to about 8.0 wt. %, or about 7.5 wt. %, based on a total weight of the composition; polymyxin B in an amount of about 0.001 wt. % to about 0.5 wt. %, about 0.005 wt. % to about 0.1 wt. %, or about 0.01 wt. % to about 0.05 wt. %, based on a total weight of the composition; and amoxicillin in an amount of about 0.01 wt. % to about 10.0 wt. %, about 0.01 wt. % to about 9.0 wt. %, about 0.01 wt. % to about 8.0 wt. %, about 0.01 wt. % to about 7.0 wt. %, about 0.01 wt. % to about 6.0 wt. %, about 0.01 wt. % to about 5.0 wt. %, 0.05 wt. % to about 4.5 wt. %, 0.1 wt. % to about 4.0 wt. %, 0.5 wt. % to about 3.5 wt. %, 1.0 wt. % to about 3.0 wt. %, 1.5 wt. % to about 2.5 wt. %, or about 2.0 wt. %, based on a total weight of the composition.

In some examples, a third composition of the present disclosure comprises cholecalciferol in an amount of about 0.001 wt. % to about 1.0 wt. %, about 0.005 wt. % to about 0.5 wt. %, about 0.01 wt. % to about 0.1 wt. %, or about 0.05 wt. %, based on a total weight of the composition; ibuprofen in an amount of about 0.01 wt. % to about 15.0 wt. %, about 0.5 wt. % to about 14.5 wt. %, about 1.0 wt. % to about 14.0 wt. %, about 1.5 wt. % to about 13.5 wt. %, about 2.0 wt. % to about 13.0 wt. %, about 2.5 wt. % to about 12.5 wt. %, about 3.0 wt. % to about 12.0 wt. %, about 3.5 wt. % to about 11.5 wt. %, about 4.0 wt. % to about 11.0 wt. %, about 4.5 wt. % to about 10.5 wt. %, about 5.0 wt. % to about 10.0 wt. %, about 5.5 wt. % to about 9.5 wt. %, about 6.0 wt. % to about 9.0 wt. %, about 6.5 wt. % to about 8.5 wt. %, about 7.0 wt. % to about 8.0 wt. %, or about 7.5 wt. %, based on a total weight of the composition; aspirin in an amount of about 0.01 wt. % to about 15.0 wt. %, about 0.5 wt. % to about 14.5 wt. %, about 1.0 wt. % to about 14.0 wt. %, about 1.5 wt. % to about 13.5 wt. %, about 2.0 wt. % to about 13.0 wt. %, about 2.5 wt. % to about 12.5 wt. %, about 3.0 wt. % to about 12.0 wt. %, about 3.5 wt. % to about 11.5 wt. %, about 4.0 wt. % to about 11.0 wt. %, about 4.5 wt. % to about 10.5 wt. %, about 5.0 wt. % to about 10.0 wt. %, about 5.5 wt. % to about 9.5 wt. %, about 6.0 wt. % to about 9.0 wt. %, about 6.5 wt. % to about 8.5 wt. %, about 7.0 wt. % to about 8.0 wt. %, or about 7.5 wt. %, based on a total weight of the composition; polymyxin B in an amount of about 0.001 wt. % to about 0.5 wt. %, about 0.005 wt. % to about 0.1 wt. %, or about 0.01 wt. % to about 0.05 wt. %, based on a total weight of the composition; amoxicillin in an amount of about 0.01 wt. % to about 10.0 wt. %, about 0.01 wt. % to about 9.0 wt. %, about 0.01 wt. % to about 8.0 wt. %, about 0.01 wt. % to about 7.0 wt. %, about 0.01 wt. % to about 6.0 wt. %, about 0.01 wt. % to about 5.0 wt. %, 0.05 wt. % to about 4.5 wt. %, 0.1 wt. % to about 4.0 wt. %, 0.5 wt. % to about 3.5 wt. %, 1.0 wt. % to about 3.0 wt. %, 1.5 wt. % to about 2.5 wt. %, or about 2.0 wt. %, based on a total weight of the composition; and levofloxacin in an amount of about 0.01 wt. % to about 10.0 wt. %, about 0.01 wt. % to about 9.0 wt. %, about 0.01 wt. % to about 8.0 wt. %, about 0.01 wt. % to about 7.0 wt. %, about 0.01 wt. % to about 6.0 wt. %, about 0.01 wt. % to about 5.0 wt. %, 0.05 wt. % to about 4.5 wt. %, 0.1 wt. % to about 4.0 wt. %, 0.5 wt. % to about 3.5 wt. %, 1.0 wt. % to about 3.0 wt. %, 1.5 wt. % to about 2.5 wt. %, or about 2.0 wt. %, based on a total weight of the composition.

In some examples, a composition of the present disclosure is a liquid and comprises at least one vitamin in an amount of about 0.001 w/v % to about 1.0 w/v %, about 0.005 w/v % to about 0.5 w/v %, about 0.01 w/v % to about 0.1 w/v %, or about 0.05 w/v %, based on a total weight of the composition; the at least one reversible COX inhibitor in an amount of about 0.01 w/v % to about 15.0 w/v %, about 0.5 w/v % to about 14.5 w/v %, about 1.0 w/v % to about 14.0 w/v %, about 1.5 w/v % to about 13.5 w/v %, about 2.0 w/v % to about 13.0 w/v %, about 2.5 w/v % to about 12.5 w/v %, about 3.0 w/v % to about 12.0 w/v %, about 3.5 w/v % to about 11.5 w/v %, about 4.0 w/v % to about 11.0 w/v %, about 4.5 w/v % to about 10.5 w/v %, about 5.0 w/v % to about 10.0 w/v %, about 5.5 w/v % to about 9.5 w/v %, about 6.0 w/v % to about 9.0 w/v %, about 6.5 w/v % to about 8.5 w/v %, about 7.0 w/v % to about 8.0 w/v %, or about 7.5 w/v %, based on a total weight of the composition; the at least one irreversible COX inhibitor in an amount of about 0.01 w/v % to about 15.0 w/v %, about 0.5 w/v % to about 14.5 w/v %, about 1.0 w/v % to about 14.0 w/v %, about 1.5 w/v % to about 13.5 w/v %, about 2.0 w/v % to about 13.0 w/v %, about 2.5 w/v % to about 12.5 w/v %, about 3.0 w/v % to about 12.0 w/v %, about 3.5 w/v % to about 11.5 w/v %, about 4.0 w/v % to about 11.0 w/v %, about 4.5 w/v % to about 10.5 w/v %, about 5.0 w/v % to about 10.0 w/v %, about 5.5 w/v % to about 9.5 w/v %, about 6.0 w/v % to about 9.0 w/v %, about 6.5 w/v % to about 8.5 w/v %, about 7.0 w/v % to about 8.0 w/v %, or about 7.5 w/v %, based on a total weight of the composition; optionally the at least one TLR inhibitor in an amount of about 0.001 w/v % to about 0.5 w/v %, about 0.005 w/v % to about 0.1 w/v %, or about 0.01 w/v % to about 0.05 w/v %, based on a total weight of the composition; and optionally each of the at least one broad-spectrum antibiotic in an amount of about 0.01 w/v % to about 10.0 w/v %, about 0.01 w/v % to about 9.0 w/v %, about 0.01 w/v % to about 8.0 w/v %, about 0.01 w/v % to about 7.0 w/v %, about 0.01 w/v % to about 6.0 w/v %, about 0.01 w/v % to about 5.0 w/v %, 0.05 w/v % to about 4.5 w/v %, 0.1 w/v % to about 4.0 w/v %, 0.5 w/v % to about 3.5 w/v %, 1.0 w/v % to about 3.0 w/v %, 1.5 w/v % to about 2.5 w/v %, or about 2.0 w/v %, based on a total weight of the composition.

In some examples, the third composition may further comprise at least one excipient selected from the group consisting of a carrier, a surfactant, a emollient, a thickening agent, a buffering agent, a sweetening agent, a flavoring agent, a coloring agent, an antioxidant, a chelating agent, a preservative, a gelling agent, and combinations thereof, as described below.

In some examples, the third composition may be used to treat periodontal disease and/or bacterial infection. Without wishing to be bound by theory, the third composition described herein may serve, in addition to the benefits described above, synergistically as bactericidal agents against stubborn infection instilled by a spectrum of markedly transformed drug-resistant pathogenic gram-negative and gram-positive bacteria firmly colonizing and disseminating state of acute and chronic periodontal diseases, neutralize proinflammatory toxins, and restore dysregulated immune imbalance and inflammatory outburst from acute and chronic periodontal diseases associated stressed conditions.

In some examples of the present disclosure, the compositions described herein, for examples, the first, second, and third compositions may comprise at least one excipient. In some examples, the excipients are at least one selected from the group consisting of a carrier, a emollient, a surfactant, a thickening agent, a buffering agent, a sweetening agent, a flavoring agent, a coloring agent, an antioxidant, a chelating agent, a preservative, a gelling agent, and combinations thereof.

In some examples, the composition may comprise a carrier. In some examples, a carrier refers to a pharmaceutically acceptable substance that is inert with respect to the active ingredient and is used to deliver, stabilize, or facilitate the administration of the active drug in a composition. Pharmaceutically acceptable carriers refer to a diluent, adjuvant, or vehicle together with which the therapeutic agent or compound is administered, and which is suitable for contacting a tissue of human and/or other animals within the scope of reasonable medical judgment, and without excessive toxicity, irritation, allergic reactions, or other problems or complications corresponding to a reasonable benefit/risk ratio. Descriptions of suitable pharmaceutically acceptable carriers, and factors involved in their selection, are found in a variety of readily available sources such as, for example, Allen, L. V, Jr. et al., Remington: The Science and Practice of Pharmacy (2 Volumes), 22nd Edition, Pharmaceutical Press (2012), and Rowe, R. C., et al., Handbook of Pharmaceutical Excipients, 6th Edition, Pharmaceutical Press (2009). In some examples, the carrier may include, but is not limited to, ion exchangers, alumina, aluminum stearate, lecithin, serum proteins, such as human serum albumin, buffering agent substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. In some examples, the compositions of the present disclosure comprise water as a carrier. In some examples, the compositions of the present disclosure comprise polyethylene glycol as a carrier. In some examples, the compositions of the present disclosure comprise a combination of water and polyethylene glycol as a carrier. In some examples, the composition may comprise a carrier in an total amount of about 30 wt. % to about 98 wt. %, based on a total weight of the composition. In other words, if more than one carrier is included, the total amount of the combined carriers is from about 30 wt. % to about 98 wt. %, based on a total weight of the composition. In some examples, the composition may comprise a carrier in an amount of about 30 wt. % to about 98 wt. %, about 30 wt. % to about 95 wt. %, about 35 wt. % to about 90 wt. %, about 40 wt. % to about 85 wt. %, about 45 wt. % to about 80 wt. %, about 50 wt. % to about 75 wt. %, about 55 wt. % to about 70 wt. %, or about 60 wt. % to about 65 wt. %, based on a total weight of the composition.

In some examples, the compositions of the present disclosure may comprise an emollient. In some examples, the composition may comprise a humectant. In some examples, the composition may comprise a substance having both emollient and humectant properties. Emollients refer to a pharmaceutically acceptable substance that is used to soften, smooth, and protect the skin by forming a protective layer on the skin's surface, thereby reducing water loss and improving skin texture. Humectants can enhance the viscosity, mouthfeel, and sweetness of the product, and may also help preserve the product from degradation or microbial contamination and keep the composition from hardening upon exposure to air. Suitable emollients and humectants include, but are not limited to, glycerin, propylene glycol, sorbitol, urea, hyaluronic acid, panthenol, sodium PCA (pyrrolidone carboxylic acid), petrolatum, mineral oil, lanolin, cocoa butter, shea butter, coconut oil, olive oil, cetyl alcohol, stearyl alcohol, cetearyl alcohol, isopropyl myristate, isopropyl palmitate, glyceryl stearate, dimethicone, cyclomethicone, jojoba oil, almond oil, and avocado oil. In some examples, the composition comprises glycerin as a humectant/emollient. In some examples, the composition may comprise at least one emollient and/or humectant in an total amount of about 50 wt. % to about 95 wt. %, about 50 wt. % to about 90 wt. %, about 50 wt. % to about 85 wt. %, about 55 wt. % to about 80 wt. %, about 60 wt. % to about 75 wt. %, or about 65 wt. % to about 70 wt. %, based on a total weight of the composition.

In some examples, the compositions of the present disclosure may comprise a surfactant. The surfactant may be selected from anionic, nonionic, amphoteric, zwitterionic, cationic surfactants, or combinations thereof. In some examples, the surfactant is a non-ionic surfactant. Examples of suitable surfactants include, but are not limited to, cetearyl alcohol, cetyl alcohol, stearyl alcohol, behenyl alcohol, lauryl alcohol, myristyl alcohol, palmityl alcohol, oleyl alcohol, sodium lauryl sulfate, sodium lauryl ether sulfate, sodium stearate, potassium cetyl sulfate, cetyltrimethylammonium bromide, benzalkonium chloride, stearalkonium chloride, polysorbate 20, polysorbate 60, polysorbate 80, sorbitan monolaurate, sorbitan monostearate, polyethylene glycol derivatives, ceteareth-20, cocamidopropyl betaine, lecithin, sodium cocoyl glycinate, and combinations thereof. In some examples, the composition includes polysorbate 60 as a surfactant. In some examples, the composition includes cetearyl alcohol as a surfactant. In some examples, the composition includes a combination of polysorbate 60 and cetearyl alcohol as a surfactant in a form of a wax, for example, Polawax™. In some examples, the composition may comprise at least one surfactant in a total amount of about 0.1 wt. % to about 10 wt. %, about 0.5 wt. % to about 9 wt. %, about 1 wt. % to about 8 wt. %, about 2 wt. % to about 7 wt. %, about 3 wt. % to about 6 wt. %, or about 4 wt. % to about 5 wt. %, based on a total weight of the composition.

In some examples, the composition may comprise a thickening agent. A thickening agent is a substance that increases the viscosity of a pharmaceutical formulation, thereby making liquids thicker or giving creams, gels, and ointments a desired texture. In some examples, the thickening agent may be, but is not limited to, hypromellose, starch, maltodextrin, xanthan gum, guar gum, tragacanth, carrageenan, sodium alginate, methylcellulose, hydroxypropyl methylcellulose, carboxymethylcellulose, carbomers, polyvinyl alcohol, polyethylene glycol (high molecular weight), hydroxyethyl cellulose, bentonite, magnesium aluminum silicate, colloidal silica, cetearyl alcohol, stearyl alcohol, cetyl alcohol, behenyl alcohol, and combinations thereof. In some examples, the composition may comprise at least one thickening agent in a total amount of about 0.1 wt. % to about 10 wt. %, about 0.5 wt. % to about 9 wt. %, about 1 wt. % to about 8 wt. %, about 2 wt. % to about 7 wt. %, about 3 wt. % to about 6 wt. %, or about 4 wt. % to about 5 wt. %, based on a total weight of the composition.

In some examples, the composition may comprise a buffering agent. A buffering agent is a substance used in pharmaceutical formulations to maintain or control the pH of a solution or preparation. In some examples, the buffering agent may be, but is not limited to, sodium phosphate, potassium phosphate, disodium hydrogen phosphate, monosodium dihydrogen phosphate, citric acid, sodium citrate, trisodium citrate dihydrate, citric acid monohydrate, acetic acid, sodium acetate, hydrochloric acid, sodium hydroxide, potassium hydroxide, tris(hydroxymethyl)aminomethane (tris), triethanolamine, boric acid, sodium borate, ammonium sulfate, potassium citrate, or a combination thereof. In some examples, the composition may comprise at least one buffering agent in a total amount of about 0.1 wt. % to about 10 wt. %, about 0.5 wt. % to about 9 wt. %, about 1 wt. % to about 8 wt. %, about 2 wt. % to about 7 wt. %, about 3 wt. % to about 6 wt. %, or about 4 wt. % to about 5 wt. %, based on a total weight of the composition.

In some examples, the composition may comprise a sweetening agent In some examples, the sweetening agent may be, but is not limited to, sucralose, sucrose, acesulfame potassium, liquid glucose, glycerin, sorbitol, liquid maltitol, saccharin sodium, aspartame, saccharin, dextrose, lactose, xylitol, maltose, levulose, sodium cyclamate, D-tryptophan, dihydrochalcones, neotame, and combinations thereof. In a case where the sweetening agent is a same compound as another excipient, for example, glycerin may act as both a humectant/emollient and a sweetening agent, the amount of the sweetening agent may be in addition to the amount of the humectant/emollient. In some examples, the composition may comprise at least one sweetening agent in a total amount of about 0.1 wt. % to about 10 wt. %, about 0.5 wt. % to about 9 wt. %, about 1 wt. % to about 8 wt. %, about 2 wt. % to about 7 wt. %, about 3 wt. % to about 6 wt. %, or about 4 wt. % to about 5 wt. %, based on a total weight of the composition.

In some examples, the composition may comprise a flavoring agent. In some examples, the flavoring agent may be, but is not limited to, essential oils including peppermint oil, spearmint oil, cinnamon oil, wintergreen oil, orange oil, lemon oil, vanilla oil, peppermint flavor, fruit flavor (e.g. peach flavor or strawberry flavor), tutti-fruity flavor, mint flavor, or a combination thereof. In some examples, the flavoring agent may include a cooling agent such as menthol, and methyl salicylate. In some examples, the composition may comprise at least one flavoring agent in a total amount of about 0.001 wt. % to about 1 wt. %, about 0.005 wt. % to about 0.5 wt. %, about 0.01 wt. % to about 0.1 wt. %, or about 0.05 wt. %, based on a total weight of the composition.

In some examples, the composition may comprise a coloring agent. In some examples, the coloring agent may be, but is not limited to, pigments, pealing agents, filler powders, talc, mica, magnesium carbonate, calcium carbonate, bismuth oxychloride, zinc oxide, titanium dioxide, iron oxide, and other materials capable of creating a visual change to the compositions. In some examples, the composition may comprise at least one coloring agent in a total amount of about 0.001 wt. % to about 1 wt. %, about 0.005 wt. % to about 0.5 wt. %, about 0.01 wt. % to about 0.1 wt. %, or about 0.05 wt. %, based on a total weight of the composition.

In some examples, the composition may comprise an antioxidant. Antioxidants are substances capable of inhibiting oxidation and that may be added to pharmaceutical products to prevent deterioration by oxidative processes. In some examples, the antioxidant may be, but is not limited to, butylatedhydroxyanisole (BHA), butylatedhydroxy toluene (BHT), sodium metabisulfite, ascorbic acid, alphatocopherol, beta-carotene, vitamin E, sodium edetate, or any combination thereof. In some examples, the composition may comprise at least one antioxidant in a total amount of about 0.1 wt. % to about 10 wt. %, about 0.5 wt. % to about 9 wt. %, about 1 wt. % to about 8 wt. %, about 2 wt. % to about 7 wt. %, about 3 wt. % to about 6 wt. %, or about 4 wt. % to about 5 wt. %, based on a total weight of the composition.

In some examples, the composition may comprise a chelating agent. Chelating agents are compounds which are used for drug stabilization, to maintain potency of active pharmaceutical ingredients and to stabilize colors and flavors. The chelating agent may be, but is not limited to citric acid, citric acid monohydrate, sodium citrate, disodium edetate, dipotassium edetate, edetic acid, fumaric acid, malic acid, phosphoric acid, sodium edetate, tartaric acid, trisodium edetate, tetrasodium pyrophosphate, gluconic acid, sodium gluconate, lactic acid, phytic acid, DTPA (diethylenetriaminepentaacetic acid), EGTA (ethylene glycol-bis(2-aminoethyl ether)-N,N,N′,N′-tetraacetic acid), sodium metabisulfite, and combinations thereof. In some examples, the composition may comprise at least one chelating agent in a total amount of about 0.1 wt. % to about 10 wt. %, about 0.5 wt. % to about 9 wt. %, about 1 wt. % to about 8 wt. %, about 2 wt. % to about 7 wt. %, about 3 wt. % to about 6 wt. %, or about 4 wt. % to about 5 wt. %, based on a total weight of the composition.

In some examples, the composition may comprise a preservative. Preservatives are compounds which are included in pharmaceutical dosage form to prevent the growth of microorganisms during the product's manufacture and shelf life. The preservative may be, but is not limited to, benzyl alcohol, chloro-butanol, chloro-cresol, alkyl esters of parabens, phenol, phenyl ethanol, benzoic acid, potassium sorbate, sodium benzoate, and combinations thereof. In some examples, the composition may comprise at least one preservative in a total amount of about 0.1 wt. % to about 10 wt. %, about 0.5 wt. % to about 9 wt. %, about 1 wt. % to about 8 wt. %, about 2 wt. % to about 7 wt. %, about 3 wt. % to about 6 wt. %, or about 4 wt. % to about 5 wt. %, based on a total weight of the composition.

In some examples, the composition may comprise a gelling agent. Gelling agents are substances used to increase the viscosity of a liquid and form a gel. The gelling agent may be, but is not limited to, polyacrylic acid, hydroxypropyl methylcellulose (HPMC), methylcellulose, sodium carboxymethylcellulose (CMC), guar gum, xanthan gum, sodium alginate, calcium alginate, gelatin, pectin, carrageenan, polyvinyl alcohol (PVA), polyethylene glycol (PEG) derivatives, and combinations thereof. In some examples, the composition may comprise at least one gelling agent in a total amount of about 0.1 wt. % to about 10 wt. %, about 0.5 wt. % to about 9 wt. %, about 1 wt. % to about 8 wt. %, about 2 wt. % to about 7 wt. %, about 3 wt. % to about 6 wt. %, or about 4 wt. % to about 5 wt. %, based on a total weight of the composition.

In some examples, the compositions of the present disclosure do not comprise opioids. In some examples, the compositions of the present disclosure are free of opioids. In some examples, the compositions of the present disclosure are essentially free of opioids. In some examples, the compositions of the present disclosure are essentially completely free of opioids. In some examples, the compositions of the present disclosure are completely free of opioids.

In some examples, the compositions of the present disclosure do not comprise ethanol. In some examples, the compositions of the present disclosure are free of ethanol. In some examples, the compositions of the present disclosure are essentially free of ethanol. In some examples, the compositions of the present disclosure are essentially completely free of ethanol.

In some examples, the composition is a liquid formulation and has a pH of about 6.5 to about 7.5. In some examples, the composition is a liquid formulation and has a pH of about 6.5 to about 7.5, about 6.6 to about 7.4, about 6.7 to about 7.3, about 6.8 to about 7.2, about 6.9 to about 7.1, about 7.0.

In some examples, the composition is a liquid formulation and has a density of about 0.7 g/mL to about 1.4 g/mL. In some examples, the composition is a liquid formulation and has a density of about 0.7 g/mL to about 1.4 g/mL, about 0.75 g/mL to about 1.35 g/mL, about 0.8 g/mL to about 1.3 g/mL, about 0.85 g/mL to about 1.25 g/mL, about 0.9 g/mL to about 1.2 g/mL, about 0.95 g/mL to about 1.15 g/mL, about 1.0 g/mL to about 1.1 g/mL, or about 1.05 g/mL.

### Formulations

In some examples, the compositions of the present disclosure, such as but not limited to the first, second, and third compositions, are formulated for administration to a subject, as described below.

In some examples, the compositions of the present disclosure can be administered through various routes, such as but not limited to oral, parenteral, or topical. The term “parenteral” as used herein includes subcutaneous, intravenous, intramuscular, intra-articular, intra-synovial, intrasternal, intrathecal, intrahepatic, intralesional and intracranial injection or infusion techniques. In preferred examples, the compositions are administered topically.

In some examples, the compositions of the present disclosure may be in the form of a suspension, a solution, an emulsion, or an emulsified suspension. In some examples, components of the composition may be dissolved in an excipient to form a solution. In some examples, particles of the components of the composition may be dispersed in an excipient to form a suspension. In some examples, the composition may be an emulsion with two immiscible liquids where one liquid is dispersed as droplets within the other and stabilized by one or more emulsifiers described herein. In some examples, the composition may be an emulsified suspension where particles of the components are dispersed within one phase of an emulsion and stabilized by one or more emulsifiers described herein. Each of the suspension, solution, emulsion, and emulsified suspension are a liquid formulation.

In some examples, the composition may be formulated as a gel, a spray, a cream, an ointment, a lotion, a foam, an oral patch, or a liquid, to be administered to a subject. Each formulation may be prepared using conventional pharmaceutical techniques and may incorporate pharmaceutically acceptable excipients as described above, as appropriate. In some examples, gels may be prepared by dispersing a gelling agent in an aqueous or hydroalcoholic vehicle, and incorporating the active ingredient therein to produce a gel formulation. In some examples, creams and ointments may be formulated as oil-in-water or water-in-oil emulsions, or as oleaginous bases, containing one or more emulsifiers, wherein the active ingredient is evenly dispersed or solubilized within the formulation. In some examples, lotions may be prepared as low-viscosity emulsions or suspensions suitable for application over large surface areas, while foams may be generated from emulsions or solutions that incorporate a gas phase to produce a stable, easily spreadable foam. In some examples, sprays may be formulated as from liquids formulations described above for administration using an aerosol or pump device, with consideration given to droplet size, uniform dosing, and stability of dispersed components. In some examples, oral patches may comprise an adhesive polymeric matrix containing the active ingredients, optionally combined with a backing layer or penetration enhancers, to provide controlled release and effective adhesion to the oral mucosa. In some examples, the formulation is a liquid as described above. In all cases, the compositions may be prepared using standard pharmaceutical procedures known in the art, such as mixing, dispersing, dissolving, emulsifying, homogenizing, or gelling, and may be adjusted for pH, viscosity, or other physicochemical properties to ensure stability, efficacy, and ease of administration.

In some examples, each of the described formulations may be combined with a toothpaste or a mouthwash. In some examples, the composition may be incorporated into a commercially available toothpaste or mouthwash by blending the active formulation with the toothpaste or mouthwash base under conditions that preserve the stability and homogeneity of both the active composition and the toothpaste or mouthwash. The blending may be performed using conventional mixing techniques known in the art, such as low-shear or high-shear mixing, to ensure uniform distribution of the active ingredient throughout the toothpaste or mouthwash. Care may be taken to maintain appropriate pH, viscosity, and rheological properties of the final product, and to prevent degradation, precipitation, or separation of the active components. The resulting medicated toothpaste or mouthwash may retain the original texture, flavor, and user-acceptable consistency of the base toothpaste or mouthwash while providing the desired pharmaceutical activity.

### Methods of Use

In some examples, the compositions of the present disclosure, such as but not limited to the first, second, and third compositions, may be used in methods of maintaining or improving oral health in a subject. In some examples, the methods comprise administering an effective amount of a composition of the present disclosure to a subject in need thereof. In some examples, the administering maintains or improves at least one oral health parameter selected from the group consisting of halitosis, tooth sensitivity, gum sensitivity, gum swelling, gum pain and inflammation, gum bleeding, plaque and tarter buildup, and combinations thereof.

In some examples, the compositions of the present disclosure, such as but not limited to the first, second, and third compositions, may be used in methods of preventing or treating periodontal disease in a subject. In some examples, the methods comprise administering an effective amount of a composition of the present disclosure to a subject in need thereof. In some examples, the administering prevents periodontal disease from forming in the subject. In some examples, the administering treats periodontal disease in the subject.

In some examples, the subject is a mammal. In some examples, the subject is a human. In some examples, the subject is a non-human animal. In some examples, the subject is a non-human mammal. In some examples, the subject is a domesticated animal, such as a dog, cat, cow, pig, horse, sheep, or goat. In some examples, the subject has a healthy oral cavity meaning, that the subject does not suffer from at least one oral health parameter selected from the group consisting of halitosis, tooth sensitivity, gum sensitivity, gum swelling, gum pain and inflammation, gum bleeding, plaque and tarter buildup, or periodontal disease and the application of a composition of the present disclosure is for maintenance of oral health. In some examples, the subject does not suffer from periodontal disease and the application of a composition of the present disclosure is for maintenance of oral health. In some examples, the subject is immunocompromised and the application of a composition of the present disclosure is for maintenance of oral health. In some examples, the subject is chronically ill and the application of a composition of the present disclosure is for maintenance of oral health. In some examples, the subject suffers from periodontal disease, as described below. In some examples, the subject suffers from autoimmune periodontal disease. In some examples, the subject is recovering from or is about to undergo an oral operative procedure, as described below.

In some examples, the subject has a periodontal disease. Periodontal disease generally has four stages. Stage I is gingivitis which is the earliest stage of periodontal disease and affects only the gums. Plaque buildup along the gumline causes redness, swelling, and bleeding, especially during brushing or flossing, and at this stage, there is no bone or tissue loss around the teeth. Stage II is early periodontitis which develops when gingivitis is left untreated and inflammation spreads below the gumline. The gums begin to pull away from the teeth, forming small periodontal pockets that trap bacteria and early bone loss may occur, although symptoms can still be mild. Stage III is moderate periodontitis which is marked by increased destruction of the supporting bone and connective tissues and periodontal pockets deepen, allowing infection to persist and worsen. Gum recession, tooth sensitivity, and occasional tooth looseness may occur. Stage IV is advanced periodontitis which involves severe bone and tissue loss around the teeth. Teeth may shift, loosen significantly, or fall out due to loss of support. In some examples, the subject has periodontal disease which is stage I, stage II, stage III, stage IV, or an intermediate thereof. In some examples, the subject has a periodontal disease selected from the group consisting of gingivitis, chronic periodontitis, aggressive periodontitis, necrotizing ulcerative gingivitis, necrotizing ulcerative periodontitis, periodontitis as a manifestation of systemic disease, refractory periodontitis, mucogingival defects, and peri-implantitis.

In some examples, the subject has gingivitis. Gingivitis is an infection characterized as short-term or acute infection and inflammation of the gum caused by bacterial nesting and growth on plaque and build-up tartar within gingival margin and pockets, and the proinflammatory toxins released therefrom. The untreated advancement of gingivitis may lead to sensitivity (hot, cold, sweet), receding gum, initiation and deepening of gingival pockets, spread of pathogenic infection to alveolar bone and pulp and setting up the stage for advanced disease state.

In some examples, the subject has periodontitis. Periodontitis is the progressive worsening of the disease as compared to gingivitis with continued deepening of gingival pocket depth, eases bacterial penetrations deep into the sulcus and instills a chronic state of infection and inflammation. This further accelerates damage of the periodontal ligaments, leaching of dentin mass (dental calcium matrix beneath exposed enamel layer), and damage of alveolar bone including dental sockets, tendon and pulp those supports dental strength and structural integrity. Periodontitis is a complex periodontal disease combination. It is a pathogenic infection, chronic inflammation and in addition an altered or compromised immune defense response resulting from failed and dysregulated chronic autoimmune host tissue reactivity. Chronic infection and inflammation with periodontal diseases gingivitis and periodontitis have negative bearings on oral health, causes excruciating oral discomfort and ultimate tooth loss. Periodontal disease is one of the major entry routes for bloodborne pathogen resulting in systemic diseases.

In some examples, the administering comprises applying the composition to an area of interest on the subject. In some examples, the administering comprises applying the composition topically to an area of interest on the subject. In some examples, the administering comprises applying the composition by injecting into an area of interest on the subject. In some examples, the area of interest is located in an oral cavity of the subject. In some examples, the administering comprises applying the composition to at least a portion of the oral cavity of the subject. In some examples, the administering comprises applying the composition topically to at least a portion of the oral cavity of the subject. In some examples, the administering comprises applying the composition by injecting into at least a portion of the oral cavity of the subject. In some examples the oral cavity includes the teeth and the periodontium which includes the gingiva (gums), cementum (covering the tooth root), alveolar bone (the jawbone socket), and the periodontal ligament (PDL), which connects the tooth root to the bone, allowing for function and cushioning. In some examples, the administering comprises applying the composition to teeth, surrounding gums, and/or dental gaps and pockets. In some examples, the administering comprises applying the composition to a portion of the gums of the subject. In some examples, the administering comprises applying the composition to a portion of the oral cavity having an infection or a disease, as described below.

In some examples, the administering comprises applying the composition to an area of interest on the subject for a period of time to exert a therapeutic effect. In some examples, the administering comprises applying the composition to an area of interest on the subject for a period of time to exert a therapeutic effect, where the therapeutic effect is maintenance or improvement of at least one oral health parameter. In some examples, the administering comprises applying the composition to an area of interest on the subject for a period of time to exert a therapeutic effect, where the therapeutic effect is prevention or treatment of periodontal disease. In some examples, the administering comprises applying the composition to an area of interest on the subject for at least about thirty seconds. In some examples, the administering comprises applying the composition to an area of interest on the subject for at least about one minute. In some examples, the administering comprises applying the composition to an area of interest on the subject for at least about two minutes, about three minutes, about four minutes, or at least about five minutes. In some examples, the administering comprises applying the composition to an area of interest on the subject for about 1 to about 20 minutes, about 2 to about 18 minutes, about 4 to about 16 minutes, about 6 to about 14 minutes, about 8 to about 12 minutes, or about 10 minutes.

In some examples, following application of the composition to the subject, the methods described herein further comprise, removing the composition from the subject. In some examples, following application of the composition to the subject, the methods described herein further comprise, removing the composition from the subject by having the subject expectorate the composition. In some examples, following application of the composition to the subject, the methods described herein further comprise, removing the composition from the subject by rinsing the mouth of the subject with a liquid and expectorating the liquid. In some examples, the liquid for rinsing is water, or any other suitable rinse (such as an alcohol free rinse) known in the art. In some examples, the composition is substantially not swallowed by the subject. In some examples, the composition is swallowed by the subject. In some examples, the application of the composition includes swishing the composition around in an oral cavity of the subject for a period of time and then expectorating the composition.

In some examples, the composition is administered to the subject daily. In some examples, the composition is administered to the subject at least once a day. In some examples, the composition is administered to the subject at least twice a day. In some examples, the composition is administered to the subject 1, 2, 3, 4, 5, 6, 7, 8, 9, or 10 times a day. In some examples, the composition is administered to the subject prior to eating. In some examples, the composition is administered to the subject after the subject's teeth are cleaned, for example, by a dental office cleaning or by brushing the teeth.

In some examples, the treatment duration with the composition may depend on the components of the composition and intended use. For example, the first composition of the present disclosure may not comprise any antibiotics, such as but not limited to the TLR inhibitor and/or the broad-spectrum antibiotic and therefore may be administered indefinitely in order to maintain or improve oral health. In some examples, the composition which does not contain antibiotics may be administered daily for at least one day, at least one week, at least one month, or at least one year, to provide maintained or improved oral health. In some examples, the first composition of the present disclosure which may not comprise any antibiotics, may be administered to the subject indefinitely as part of a daily oral hygiene routine. In some examples, the oral hygiene routine may include, but is not limited to, routine tooth brushing, flossing, and/or applying a mouthwash, at least once a day. In some examples, the composition is applied to the subject before, during or after the oral hygiene routine. In some examples, the composition is included in the toothpaste or mouthwash, as described above. In some examples, the first composition of the present disclosure, is administered to the subject as part of a routine cleaning at a dental office. In some examples, the subject undergoes a routine cleaning at a dental office at least once a year, or 2, 3, 4, 5, 6, 7, 8, 9, or 10 times a year. In some examples, the composition is applied to the subject in the routine cleaning at a dental office, by a dental professional, including but not limited to a dentist, dental surgeon, oral and maxillofacial surgeon, periodontist, endodontist, orthodontist, prosthodontist, pediatric dentist, public health dentist, oral pathologist, dental hygienist, dental therapist, or dental assistant.

In some examples, the treatment duration with the composition may depend on the components of the composition and intended use. For example, the second and/or third compositions of the present disclosure which may comprise antibiotics, such as but not limited to the TLR inhibitor and/or the broad-spectrum antibiotic, should be administered as according to a standard administration of said antibiotic, as would be understood by one of ordinary skill in the art. In some examples, a composition containing an antibiotic is administered for a therapeutically effective duration. In some examples, the therapeutically effective duration is less than about one month, less than about three weeks, about 14 days, about 12 days, about 10 days, or about 7 days. In some examples, the therapeutically effective duration is at least once a day for a length of time which is sufficient to treat the bacterial infection. In some following treatment of the bacterial infection, the administering of the composition containing an antibiotic is ceased, and administration of the first composition without the antibiotics may be started. In some examples, during the therapeutically effective duration, the composition may be administered as part of an oral hygiene routine which may include, but is not limited to, routine tooth brushing, flossing, and/or applying a mouthwash, at least once a day. In some examples, the composition is applied to the subject before, during or after the oral hygiene routine. In some examples, the composition is included in the toothpaste or mouthwash, as described above. In some examples, the second or third composition of the present disclosure, is administered to the subject as part of a dental office visit. In some examples, the composition is applied to the subject at the dental office, by a dental professional, including but not limited to a dentist, dental surgeon, oral and maxillofacial surgeon, periodontist, endodontist, orthodontist, prosthodontist, pediatric dentist, public health dentist, oral pathologist, dental hygienist, dental therapist, or dental assistant.

In some examples, the composition is administered to the subject as part of pre-operative care. In some examples, the composition may prevent infection during the operative process. In some examples, the composition is administered to the subject as part of post-operative care. In some examples, the composition may prevent infection after the operative process. In some examples, the composition is administered to the subject as part of pre or post-operative care for a procedure selected from the group consisting of scaling and root planning, osseous surgery, root canal, periodontal pocket reduction, flap surgery, soft tissue gum graft, bone graft, LANAP (Laser-Assisted New Attachment Procedure), and dental implant surgery.

In some examples, administration of a composition of the present disclosure decreases inflammation in a subject. In some examples, administration of a composition of the present disclosure decreases inflammation in a subject as evidenced by a decrease in levels of at least one selected from the group consisting of prostaglandin E2 (PGE2) and pro-inflammatory cytokines. In some examples, administration of a composition of the present disclosure decreases inflammation in a subject as evidenced by a decrease in levels of pro-inflammatory cytokines. In some examples, administration of a composition of the present disclosure decreases inflammation in a subject as evidenced by a decrease in levels of at least one selected from the group consisting of PGE2, IL-1a, IL-1b, TNFa, IFNg, IL-6, and IL-8. In some examples, administration of a composition of the present disclosure improves or maintains an oral health of a subject by decreasing levels of at least one pro-inflammatory cytokine selected from the group consisting of IL-1a, IL-1b, TNFa, IFNg, IL-6, and IL-8, while maintaining a larger amount of the pro-regulatory cytokines (IL-10, IL-22). In some examples, on administering a composition of the present disclosure, levels of pro-inflammatory cytokines (IL-1a, IL-1b, TNFa, IFNg, IL-6, IL-8) are decreased in a higher amount than pro-regulatory cytokines (IL-10, IL-22).

In some examples, the composition is administered in an amount of about 25 mg to about 150 mg of the total composition per application to the subject. In some examples, the composition is administered in an amount of about 25 mg to about 150 mg, about 30 mg to about 145 mg, about 35 mg to about 140 mg, about 40 mg to about 135 mg, about 45 mg to about 130 mg, about 50 mg to about 125 mg, about 55 mg to about 120 mg, about 60 mg to about 125 mg, about 65 mg to about 120 mg, about 70 mg to about 115 mg, about 75 mg to about 110 mg, about 80 mg to about 105 mg, about 85 mg to about 100 mg, or about 90 mg to about 95 mg, of the total composition per application to the subject. In some examples, the composition is a liquid and is administered in an amount of about 10 μL to about 100 mL per application. In some examples, the composition is a liquid and is administered in an amount of about 10 μL to about 100 mL, about 50 μL to about 50 mL, about 100 μL to about 1 mL, or about 500 μL, per application.

In some examples, a composition of the present disclosure may be used in preventing periodontal pathogen invasion, microbial replication, infection, inflammation, neutralization of invading pathogen released/derived toxins, mitigate periodontal disease associated symptoms, reduce diseases progression recurrences and severity, support wound healing and regenerative system repair, and rebalancing of osteoclast osteoblast activity in conserving periodontal ligaments tendons and alveolar bone integrity. In some examples, a composition of the present disclosure may be used in preventing periodontal pathogen invasion, impede microbial surface adherence and proliferation within gingival margin and pockets, deter plaque and tartar buildup; support alveolar bone remodeling, boost damaged tissue and ligament repair, provide oral immune support and strengthen periodontium system agility against diseases and non-disease associated stressed conditions.

In some examples, a composition of the present disclosure, such as the third composition, may therapeutically address and regulate the adverse issues with unremitting red zone microbial infections such as but not limited to, families of gram-positive and gram-negative bacteria, Porphyromonas gingivalis, Tannerella forsythia, Treponema denticola, Filifactor alocis, Synergistetes, Peptostreptococcaceae, Actinomyces actinomycetemcomitans, Fusobacterium nucleatum, Streptococcus mutans, Streptococcus sobrinus, Lacticaseibacillus casei, and the like. Microbial pathogens like these are known to evade the immune response and are largely persistent throughout asymptomatic to latent, to severe stages of acute-to-chronic periodontal diseases. In some examples, the periodontal disease stems from recurrent pathogenic infection and disease relapse, breakdown of host immune defense, adoption of a corrupt or dysregulated and compromised innate and adaptive immune system, severe gingival tissue damage, bone resorption, tooth mobility leading to tooth loss, and possible bloodborne systemic disease transmission, where such infection and disease state are associated with chronic infection from a network of diverse invasive often antibiotic-resistant microbial pathogens, such as but not limited to, Actinobacillus actinomycetemcomitans, Porphyromonas gingivalis, Actinomyces naeslundii, Streptococcus mutans, and the like.

It should also be understood that a specific dosage and treatment regimen for any particular patient will depend upon a variety of factors, including the activity of the specific compound employed, allergic sensitivity if any to the active components of the composition, the age, body weight, general health, sex, diet, time of administration, rate of excretion, drug combination, and the judgment of the treating physician and the severity of the particular disease or disorder being treated. The amount of a compound of the present disclosure in the composition will also depend upon the particular compound in the composition.

### Methods of Making

In some examples, the compositions of the present disclosure may be provided in the form of a suspension, a solution, an emulsion, or an emulsified suspension and may comprise the components as described above in combination with one or more pharmaceutically acceptable excipients. The selection of excipients may depend on factors such as the intended route of administration, dosage form, and stability requirements.

In some examples, the compositions of the present disclosure may be prepared by combining the components as described above with one or more excipients in a suitable mixing vessel using conventional formulation techniques. For solution formulations, the components as described above may be dissolved in an aqueous or non-aqueous solvent system under agitation, optionally with heating, to form a substantially clear and homogeneous solution. One or more excipients may be added before, during, or after dissolution of the components as described above.

In some examples, in which the compositions of the present disclosure are a suspension, the components as described above may be dispersed as solid particles within a liquid medium. The liquid medium may comprise water or another pharmaceutically acceptable solvent, along with one or more surfactants to promote uniform dispersion and inhibit particle aggregation or settling. Mixing may be performed using mechanical agitation, high-shear mixing, or milling techniques to achieve a desired particle size distribution and suspension stability.

In some examples, in which the compositions of the present disclosure are an emulsion, the composition may be prepared by separately forming an oil phase and an aqueous phase. The oil phase may comprise one or more lipophilic excipients, while the aqueous phase may comprise water and one or more hydrophilic excipients. One or more may be included in either or both phases. The phases may be heated independently, optionally to substantially similar temperatures, and subsequently combined under agitation to form an emulsion.

In some examples, heating may be applied during formulation to facilitate dissolution, dispersion, or emulsification of the components or excipients. Heating may be conducted at temperatures above ambient and below the degradation temperature of the components, for example from about 25° C. to about 80° C. Following mixing, the composition may be cooled under continued agitation. Additional processing steps, such as pH adjustment, filtration, homogenization, deaeration, or sterilization, may be performed as appropriate for the intended use.

It will be understood that the order of addition, processing conditions, and component concentrations may be varied without departing from the scope of the invention, and that the described formulation methods are provided for illustrative purposes only.

### EXAMPLES

### Example 1

Formulation 1 was prepared in a sterile environment as follows. Powders of aspirin, ibuprofen, and cholecalciferol were mixed to form a homogeneous powder. Each of the powders were moisture free. To the homogeneous powder, purified water, polyethylene glycol (PEG) (PEG-400 and PEG-3350), polysorbate 60, hypromellose, maltodextrin, and starch were added in sequence while high speed mixing at about 40° C. Then glycerin was added and mixed to form an emulsified liquid suspension.

The active components were dispersed in the emulsified liquid suspension. On standing at room temperature for more than 24 hours, Formulation 1 may partially separate with the formation of precipitates, however, agitation provided the emulsified liquid suspension again.

Optionally flavoring agent and coloring agents were added. Glycerin was added in an amount to reach a final batch weight (volume). The amounts of the active components in Formulation 1 are shown in Table 1 and the amounts of the excipients are shown in Table 1A.

TABLE 1
Amounts of Active Components in Formulation 1

| Component | Amount | |
|---|---|---|
| Aspirin | 0.64 | w/v % |
| Ibuprofen | 6.3 | w/v % |
| Cholecalciferol (~1,350,000 IU/100 mL) | 0.034 | w/v % |

TABLE 1A
Amounts of Excipients in Formulation 1

| Component | Amount | |
|---|---|---|
| Water | 4.38 | mL |
| PEG-400 | 5.84 | mL |
| PEG-3350 | 300 | mg |
| Polysorbate 60 | 730 | mg |
| Hypromellose | 150 | mg |
| KOH | 240 | mg |
| Mint and Spearmint Flavoring Agent | 1.5 | ml |
| Maltodextrin and Starch | 16.7 | gm |

| | |
|---|---|
| Glycerin | Amount to 100 mL |

Formulation 2 was prepared in a sterile environment as follows. Powders of aspirin, ibuprofen, and cholecalciferol were mixed to form a homogeneous powder. Each of the powders were moisture free. To the homogeneous powder, purified water, polyethylene glycol (PEG) (PEG-400 and PEG-3350), polysorbate 60, hypromellose, maltodextrin, and starch were added in sequence while mixing at about 40° C. Then glycerin was added and mixed to form an emulsified liquid suspension. Polymyxin B was then added while stirring.

The active components were dispersed in the emulsified liquid suspension. On standing at room temperature for more than 24 hours, Formulation 2 may partially separate with the formation of precipitates, however, agitation provided the emulsified liquid suspension again.

Optionally flavoring agent and coloring agents were added. Glycerin was added in an amount to reach a final batch weight. The amounts of the active components in Formulation 2 are shown in Table 2. The amounts of the excipients were the same as Formulation 1 other than the variable amount of glycerin.

TABLE 2
Amounts of Active Components in Formulation 2

| | |
|---|---|
| Component | Amount |

| | | |
|---|---|---|
| Aspirin | 0.64 | w/v % |
| Ibuprofen | 6.3 | w/v % |
| Cholecalciferol (~1,350,000 IU/100 mL) | 0.034 | w/v % |

| | |
|---|---|
| Polymyxin B | 70,600 IU/ml or |
| | 0.35 w/v % |

Formulations 3 and 4 were prepared in a sterile environment as follows. Powders of aspirin, ibuprofen, and cholecalciferol were mixed to form a homogeneous powder. Each of the powders were moisture free. To the homogeneous powder, purified water, polyethylene glycol (PEG) (PEG-400 and PEG-3350), polysorbate 60, hypromellose, maltodextrin, and starch were added in sequence while mixing at about 40° C. Then glycerin was added and mixed to form a emulsified liquid suspension. Polymyxin B was then added while stirring. Antibiotics amoxicillin and optionally levofloxacin were then added while stirring.

The active components were dispersed in the emulsified liquid suspension. On standing at room temperature for more than 24 hours, Formulation 3 or Formulation 4 may partially separate with the formation of precipitates, however, agitation provided the emulsified liquid suspension again.

Optionally flavoring agent and coloring agents were added. Glycerin was added in an amount to reach a final batch weight. The amounts of the active components in Formulations 3 and 4 are shown in Tables 3 and 4, respectively. The amounts of the excipients were the same as Formulation 1 other than the variable amount of glycerin.

TABLE 3
Amounts of Active Components in Formulation 3

| | |
|---|---|
| Component | Amount |

| Aspirin | 0.64 | w/v % |
|---|---|---|
| Ibuprofen | 6.3 | w/v % |
| Cholecalciferol (~1,350,000 IU/100 mL) | 0.034 | w/v % |
| Polymyxin B | 70,600 IU/ml or | |
| | 0.35 w/v % | |
| Amoxcillin | 2.0 | w/v % |

TABLE 4
Amounts of Active Components in Formulation 4

| | |
|---|---|
| Component | Amount |

| Aspirin | 0.64 | w/v % |
|---|---|---|
| Ibuprofen | 6.3 | w/v % |
| Cholecalciferol (~1,350,000 IU/100 mL) | 0.034 | w/v % |
| Polymyxin B | 70,600 IU/ml or | |
| | 0.35 w/v % | |
| Amoxcillin | 1.0 | w/v % |
| Levofloxacin | 1.0 | w/v % |

Comparative formulations C0, C1, C2, and C3 were prepared similarly to the methods for Formulations 1-4. Formulation C0 included active components aspirin and ibuprofen. Formulation C1 included active components amoxicillin and optionally levofloxacin, cholecalciferol, and polymyxin B, without aspirin or ibuprofen. Formulation C2 included active components amoxicillin and optionally levofloxacin, aspirin, ibuprofen, and polymyxin B, without cholecalciferol. Formulation C3 included active components amoxicillin and optionally levofloxacin, cholecalciferol, aspirin, and ibuprofen, without polymyxin B. The amounts of each of the active components were the same in comparative formulations C0, C1, C2, and C3 as the Formulations 1-4. The amounts of the excipients were the same as Formulation 1 other than the variable amount of glycerin.

### Example 2

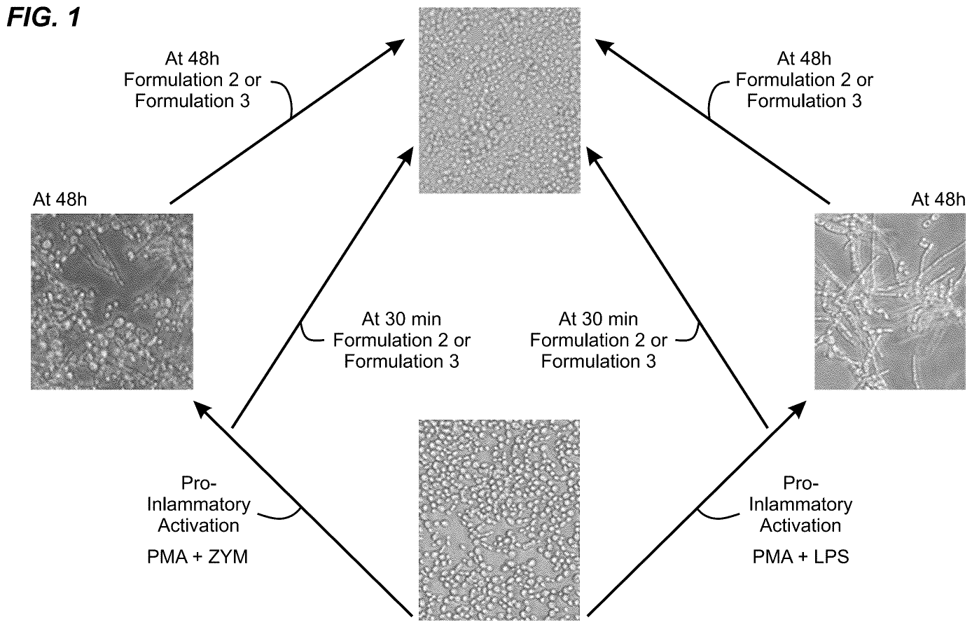

Formulation 2 and Formulation 3, as described in Example 1, were tested in vitro for efficacy in treating periodontal disease. The results are shown in FIG. 1.

Periodontal diseased cells were cultured from a human patient having end stage periodontitis as shown in the bottom middle culture of FIG. 1. The rested cells were exposed to pro-inflammatory stimuli that is phorbol 12-myristate 13-acetate (PMA) (an activator of protein kinase C (PKC)) in combination with either salmonella lipopolysaccharide (a TLR-4 agonist, labeled as LPS) or zymosan (a TLR-2 agonist, labeled as ZYM). The cells were exposed to the pro-inflammatory stimuli for 30 minutes or 48 hours.

The cells exposed to PMA and ZYM after 48 hours are shown in the middle left cell culture of FIG. 1 and exhibited severe inflammation and a change in morphology. These results were similar for the cells exposed to PMA and LPS after 48 hours as shown in the middle right cell culture in FIG. 1.

Following exposure of the cells to the pro-inflammatory stimuli for 30 minutes or 48 hours, the cells were treated with Formulation 2 or Formulation 3. As shown in the top middle cell culture of FIG. 1, Formulation 2 and Formulation 3 reversed the inflammation and morphology changes in the cells to the pre-inflammatory resting state, regardless of whether the inflammation was 30 minutes or 48 hours. This suggests that Formulation 2 and Formulation 3 provide protective and regenerative recovery at a cellular level of inflammatory disease.

### Example 3

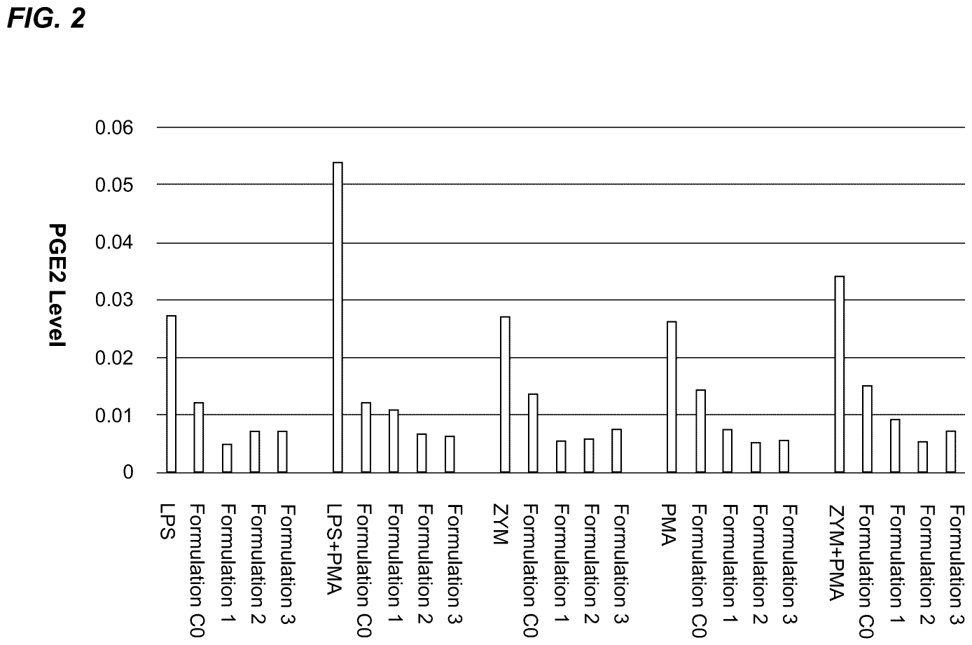

Formulations 1-3, as described in Example 1, were tested in vitro for efficacy in treating periodontal disease. The results are shown in FIG. 2.

FIG. 2 illustrates the anti-inflammatory properties of Formulations 1-3 using an in vitro cellular periodontal disease model. Cells were cultured from a human patient having end stage periodontitis. The periodontal cells were exposed to pro-inflammatory stimuli PMA, LPS, or ZYM, alone or in combination (LPS+PMA or ZYM+PMA). Following exposure of the cells to the pro-inflammatory stimuli for 30 minutes, the cells were treated with Formulation C0, Formulation 1, Formulation 2, and Formulation 3. The cells were each exposed to Formulation C0, Formulation 1, Formulation 2, and Formulation 3 for 40 hours, after which the status of the pro-inflammatory and regulatory response factors (in combined cellular and released) were determined. All other conditions were maintained between samples.

The pro-inflammatory and regulatory response was measured by the prostaglandin E2 (PGE2) levels in the cells. PGE2 is a lipid mediator that increases during inflammation, injury, and disease, acting as a key modulator in pain, fever, and immune responses. As shown in FIG. 2, following pro-inflammatory stimulation with PMA, LPS, or ZYM, alone or in combination, the cells exhibited high PGE2 levels.

Following treatment with Formulation C0 the inflammation in the cells decreased. However, cell treatment with Formulation 1, Formulation 2, or Formulation 3 provided a larger inflammatory decrease than Formulation C0 due to the synergistic combination of the components which are absent from Formulation C0. Formulation 2 exhibited an improved inhibitory response profile against robust inflammatory stimulation of LPS+PMA and ZYM+PMA combinations.

This suggests that Formulations 1-3 are effective in controlling inflammatory COX-2 enzyme activity and inflammatory mediator PGE-2. This is in addition to the reversal of stressed cell morphology from inflammation to the non-inflammatory resting state as described in Example 2.

### Example 4

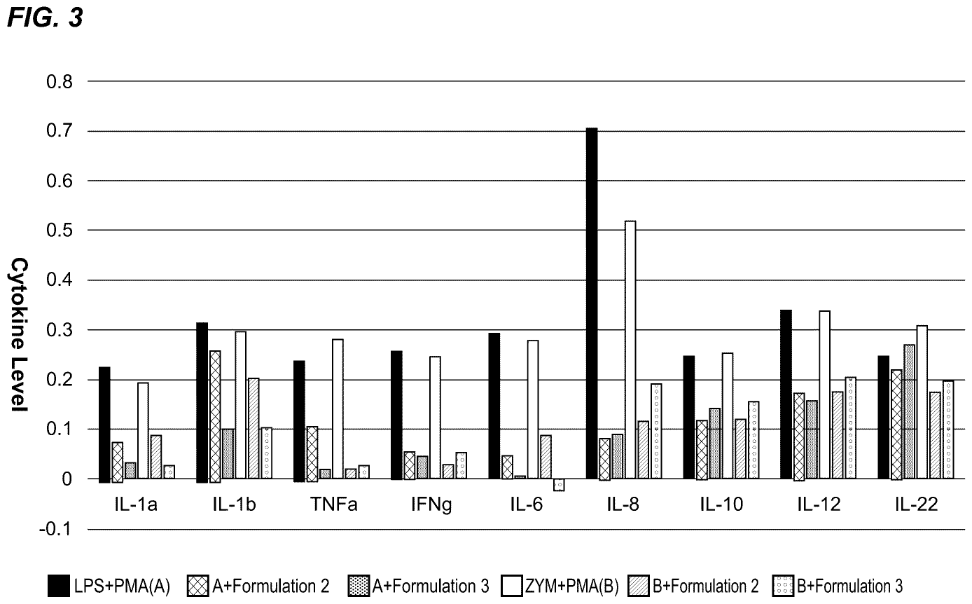

Formulation 2 and Formulation 3, as described in Example 1, were tested in vitro for efficacy in treating periodontal disease. The results are shown in FIG. 3.

FIG. 3 illustrates the anti-inflammatory properties of Formulation 2 and Formulation 3 using an in vitro cellular periodontal disease model. Cells were cultured from a human patient having end stage periodontitis. The periodontal cells were exposed to pro-inflammatory stimuli PMA in combination with LPS or ZYM (LPS+PMA or ZYM+PMA). Following exposure of the cells to the pro-inflammatory stimuli for 30 minutes, the cells were treated with Formulation 2 or Formulation 3. The cells were each exposed to Formulation 2 or Formulation 3 for 40 hours, after which the status of the pro-inflammatory and regulatory response factors (in combined cellular and released) were determined. All other conditions were maintained between samples.

The pro-inflammatory and regulatory response was measured by the cytokine inhibition profile for both pro-inflammatory cytokines (IL-1a, IL-1b, TNFa, IFNg, IL-6, IL-8) and pro-regulatory cytokines (IL-10, IL-22) and IL-12. As shown in FIG. 3, following pro-inflammatory stimulation the cells, with LPS+PMA or ZYM+PMA, exhibited high cytokine levels.

Following treatment with Formulation 2 or Formulation 3 the levels of the cytokines decreased, as shown in FIG. 3, with increased inhibition of the inflammatory cytokine levels compared to the regulatory cytokines. This suggests that Formulation 2 and Formulation 3 are effective in regulating a large number of pro-inflammatory cytokines, which are otherwise detrimental and increase worsening of both acute and chronic inflammatory periodontal diseases. In addition, Formulation 2 and Formulation 3 allow survival of a substantial level of pro-regulatory cytokines such as IL-10 and IL-22. Without wishing to be bound by theory, it is believed that decreasing inflammatory cytokine levels while maintaining or only slightly decreasing regulatory cytokine levels, enables the reversal and resolution of the inflammatory state.

### Example 5

Formulations 1-4, as described in Example 1, were tested for antimicrobial activity. The results are shown in Tables 5 and 6.

Each of Formulations 1~4 were tested against a panel of gram-positive and gram-negative bacteria linked to aggressive periodontal diseases, including P. gingivalis, P. anaerobius, P. intermedia, B. fragilis, S. mutans, L. casei, and S. pneumoniae. The antimicrobial activity was measured as MIC (Minimum Inhibitory Concentration) which is the lowest drug level stopping visible microbial growth and MBC (Minimum Bactericidal Concentration) which is the lowest concentration killing ≥99.9% of microbes.

Each of Formulations 1~4 were diluted 10× to form a prediluted stock and then 2-fold serial dilutions were prepared starting at 50% down to 0.025% dilution. The testing was performed following a standard MIC/MBC assay American Type Culture Collection (ATCC) protocol (agar or broth micro-dilution method) standardized by the Clinical and Laboratory Standards Institute (CLSI). The agar micro dilution assay was used for P. gingivalis (ATCC 33277), P. anaerobius (ATCC 27337), P. intermedia (ATCC 49046), and B. fragilis (ATCC 25285), and the broth micro dilution assay was used for S. mutans (ATCC 31341), L. casei (ATCC 334), and S. pneumoniae (ATCC49619). The numbers reported in Table 5 are the percent dilution of each test sample at which they achieved MIC and MBC activity against individual pathogens. The lowest possible concentrations were limited to 0.025% in agar micro dilution assay and 0.05% in the broth micro dilution assay, therefore these MBC and MIC values may be less than 0.025% or 0.05%. Formulations 1-4 were compared against a control containing only the excipients. Formulations 1-4 are labeled as F1-4 in Table 5. The MIC and MBC are reported in %.

TABLE 5
Antimicrobial Activity of Formulations

| | P. gingivalis | | P. anaerobins | | P.intermedia | | B. fragilis | | S. mutans | | L. casei | | S. pneumoniae | |
|---|---|---|---|---|---|---|---|---|---|---|---|---|---|---|
| Pathogen | MIC | MBC | MIC | MBC | MIC | MBC | MIC | MBC | MIC | MBC | MIC | MBC | MIC | MBC |
| Cal | 6.25 | 6.25 | 12.5 | 12.5 | 12.5 | 12.5 | 25 | 25 | 25 | 50 | >50 | >50 | 6.25 | 6.25 |
| F1 | 0.78 | 0.78 | 0.78 | 0.78 | 0.78 | 0.78 | 1.56 | 1.56 | 0.78 | 1.60 | 12.50 | 12.50 | <0.05 | <0.05 |
| F2 | <0.025 | <0.025 | 0.20 | 0.20 | <0.025 | *0.025 | 0.39 | 0.39 | 0.10 | 0.10 | 0.78 | 0.78 | <0.05 | :0.05 |
| F3 | <0.025 | <0.025 | <0.025 | <0.025 | <0.025 | <0.025 | 0.78 | 0.78 | <0.05 | <0.05 | <0.05 | <0.05 | <0.05 | <0.05 |
| F4 | <0.025 | <0.025 | <0.025 | <0.025 | <0.025 | <0.025 | <0.025 | <0.025 | <0.05 | :0:05 | <0.05 | <0.05 | <0.05 | <0.05 |

As shown in Table 5, each of Formulations 1-4 exhibited antibacterial activity against the tested pathogens, as compared to the control. Formulation 4 exhibited the highest activity against each pathogen. This suggests that Formulations 1-4 may be used in treating some infections caused by bacteria in periodontal disease.

The MIC and MBC potency of each active ingredient was also tested. Table 6 shows the active components of Formulations 1-4 and percent dilution to concentration conversion of each active ingredient in Formulations 1-4 representing the observed MIC and MBC activity. The amounts are reported in IU/mL for polymyxin B and μg/mL for all other components.

TABLE 6
Antimicrobial Activity of Each Active Ingredient in Formulations 1-4

| | | | Formulation | | Formulation | | Formulation | | Formulation | |
|---|---|---|---|---|---|---|---|---|---|---|
| Component | Control | | 1 | | 2 | | 3 | | 4 | |
| Dilution (%) | 0.025 | 0.05 | 0.025 | 0.05 | 0.025 | 0.05 | 0.025 | 0.05 | 0.025 | 0.05 |
| Aspirin | 0 | 0 | 0.08 | 0.3 | 0.08 | 0.3 | 0.08 | 0.3 | 0.08 | 0.3 |
| Ibuprofen | 0 | 0 | 0.8 | 3 | 0.8 | 3 | 0.8 | 3 | 0.8 | 3 |
| Cholecalciferol | 0 | 0 | 0.01 | 0.017 | 0.01 | 0.017 | 0.01 | 0.017 | 0.01 | 0.017 |
| Polymyxin B | 0 | 0 | 0 | 0 | 0.9 | 2 | 0.9 | 2 | 0.9 | 2 |
| Amoxcillin | 0 | 0 | 0 | 0 | 0 | 0 | 1 | 2 | 0.5 | 1 |
| Levofloxacin | 0 | 0 | 0 | 0 | 0 | 0 | 0 | 0 | 0.5 | 1 |

The data in both Tables 5 and 6 suggest that both Formulations 3 and 4 are effective in active infection control at the lowest concentration of antibiotics and are effective against a wide range of gram-negative and gram-positive pathogens in periodontal diseases.

### Example 6

Formulation 2 and Formulation 3, as described in Example 1, were tested for therapeutic potency, such as anti-infection and anti-inflammatory properties in treating periodontal disease. The results are shown in Tables 7-9.

Formulation 2 and/or Formulation 3 was administered following a preset treatment program to volunteering human subjects suffering from high-risk stage-III, stage-IV and worsening periodontitis disease and other long-term periodontal complications, under the guidance of a licensed medical professional and a qualified periodontal-specialist.

There were two cohorts receiving: 1) Formulation 2 only and 2) Formulation 2 and Formulation 3. The formulations were topically administered and retained for 2-5 minutes per application in the oral cavity in the surroundings of the periodontium via natural or non-invasive gentle mechanical whole mouth dispersion. The amount of the formulation applied was 50 to 100 μL per application twice per day for a minimum pre-set number of days.

A pre- and post-treatment score in advancement or reversal of periodontal disease stage and symptoms were assessed as a measure of “qualitative status” on a scale of 1-10 with 10 being severe, for bad breath (A), plaque and tartar buildup (B), gum pain and toothache (C), gum redness and swelling (active infection) (D), frequent gum bleeding (E), bleeding on probing (F), and hot and cold sensitivity/discomfort (G), as shown in Table 8. Each patient is summarized in Table 7, with Formulation 2 and Formulation 3 labeled as F2 and F3, respectively.

TABLE 7
Patient Description

| | | | Pre-treatment | Days of | # of Teeth | |
|---|---|---|---|---|---|---|
| Patient | Age | Sex | disease status | Routine | Pre- | Post- |
| 1 | 60 | F | Post-LANAP | 7 days of F3 | 27 | 27 |
| | | | complication | 22 days of F2 | | |
| 2 | 43 | M | Stage III-IV | 7 days of F3 | 26 | 26 |
| | | | | 4 months of F2 | | |
| 3 | 42 | F | Stage II-III | 7 days of F3 | 28 | 28 |
| | | | | 22 days of F2 | | |
| 4 | 38 | M | Stage IV | 7 days of F3 | 28 | 28 |
| | | | | 50 days of F2 | | |
| 5 | 32 | F | Tobacco use and | 100 days of F2 | 24 | 24 |
| | | | orthodontic brace | | | |
| | | | defect | | | |
| 6 | 32 | F | Tobacco use, oral | 36 days of F2 | 28 | 28 |
| | | | hygiene, veneer, | | | |
| | | | Stage III | | | |
| 7 | 29 | M | Tobacco use, Stage | 7 days of F3 | 30 | 30 |
| | | | IV | 60 days of F2 | | |
| 8 | 60 | M | Osseous defect | 7 days of F3 | 22 | 22 |
| | | | concavities | 1 year 4 | | |
| | | | | months of F2 | | |
| 9 | 34 | M | Juvenile periodontitis, | 7 days of F3 | 28 | 28 |
| | | | post gum graft, Stage | 1 year of F2 | | |
| | | | II-IV | | | |

TABLE 8
Pre- And Post- Treatment Score in of Periodontal Disease Stage and Symptoms
for Each Patient

| | (A) | | (B) | | (C) | | (D) | | (E) | | (F) | | (G) | |
|---|---|---|---|---|---|---|---|---|---|---|---|---|---|---|
| Patient | pre | post | pre | post | pre | post | pre | post | pre | post | pre | post | pre | post |
| 1 | 8 | 0-1 | 9 | 3 | 0-2 | 0-2 | 0-2 | 0-2 | 0-1 | 0-1 | 4 | 0-1 | 8 | 0-1 |
| 2 | 8 | 3 | 9 | 2 | 9 | 0-1 | 9 | 0-1 | 9 | 0 | 10 | 2 | 9 | 0-1 |
| 3 | 7 | 2 | 4 | 0-1 | 9 | 0-1 | 8 | 0-1 | 0-1 | 0-1 | 10 | 0-1 | 9 | 0-1 |
| 4 | 9 | 3 | 8 | 3 | 10 | 2 | 9 | 0-2 | 9 | 0-1 | 10 | 4 | 8 | 2 |
| 5 | 8 | 1 | 8 | 2 | 9 | 1 | 8 | 2 | 8 | 0-1 | 8 | 2 | 9 | 2 |
| 6 | 7 | 3 | 8 | 2 | 8 | 2 | 9 | 2 | 8 | 0-1 | 10 | 4 | 6 | 2 |
| 7 | 9 | 4 | 8 | 2 | 9 | 2 | 9 | 2 | 9 | 1 | 10 | 5 | 10 | 2 |
| 8 | 8 | 2 | 9 | 1 | 0-2 | 0-2 | 10 | 0-1 | 8 | 0-1 | 6 | 2 | 10 | 3 |
| 9 | 6 | 1 | 9 | 2 | 4 | 1 | 9 | 2 | 7 | 0-2 | 10 | 4 | 6 | 0-1 |

In addition, a 3-point average semi-quantitative measure of lingual and buccal perio-pocket depth was measured for each patient pre and post treatment, as shown in Table 9.

TABLE 9
Pre- And Post- Treatment Periodontal Pocket Depth for Each Patient

| | Sum of average of each 3- | | | | Reduction of pocket depth (mm) | | | |
|---|---|---|---|---|---|---|---|---|
| | point measurement of total | | | | | | Top single teeth | |
| | pocket depth (mm) | | | | Total recovery | | pocket recovery | |
| | Lingual | | Buccal | | Lingual | Buccal | Lingual | Buccal |
| Patient | pre | post | pre | post | post | post | post | post |
| 1 | 82 | 77 | 77 | 64 | 5 | 13 | 1.33 | 1.33 |
| 2 | 97 | 79 | 88 | 77 | 18 | 11 | 1.33 | 1.33 |
| 3 | 97 | 74 | 102 | 78 | 23 | 24 | 1.33 | 1.7 |
| 4 | 123 | 108 | 143 | 124 | 15 | 19 | 1.7 | 1.7 |
| 5 | 76 | 68 | 75 | 70 | 8 | 5 | 1.33 | 0.7 |
| 6 | 85 | 80 | 89 | 77 | 5 | 12 | 1 | 1 |
| 7 | 145 | 122 | 156 | 120 | 23 | 36 | 1.33 | 2 |
| 8 | 76 | 74 | 77 | 69 | 2 | 8 | 1.33 | 1.33 |
| 9 | 111 | 104 | 107 | 94 | 7 | 13 | 1.33 | 1 |

As shown in Table 8, patients receiving Formulation 2 only, or Formulation 2 and Formulation 3, exhibited major long-term stable recovery on all parameters of “qualitative status”. In addition, all patients also exhibited improvement in perio-pocket depth, as shown in Table 9. Patients receiving both Formulation 2 and Formulation 3 exhibited faster recovery.

Perio-pocket depth reduction varied between each patient and was dependent on the perio-pocket depth pre-treatment status. Higher perio-pocket depths, for example, greater than 4 millimeter/teeth had the greatest improvement. Top whole-mouth perio-pocket depth improvement ranged from 36 millimeter (buccal) to 23 millimeter (lingual) in patients with 30 or less teeth and receiving both Formulation 2 and Formulation 3. Patients that received only Formulation 2 or that had low perio-pocket depth pre-treatment exhibited improvement in the quality of the periodontium (gum-tone, pain, bleeding, sensitivity, plaque and tarter buildup, strengthening of root and chewing ability), but exhibited perio-pocket depth improvement in the single digit range (5-12 millimeter) with 24 and higher number of teeth during the study. This suggests that application of the easy to use non-invasive, non-toxic, formulations provided lasting resolution and reversal of inflammatory periodontal diseases.

### Example 7

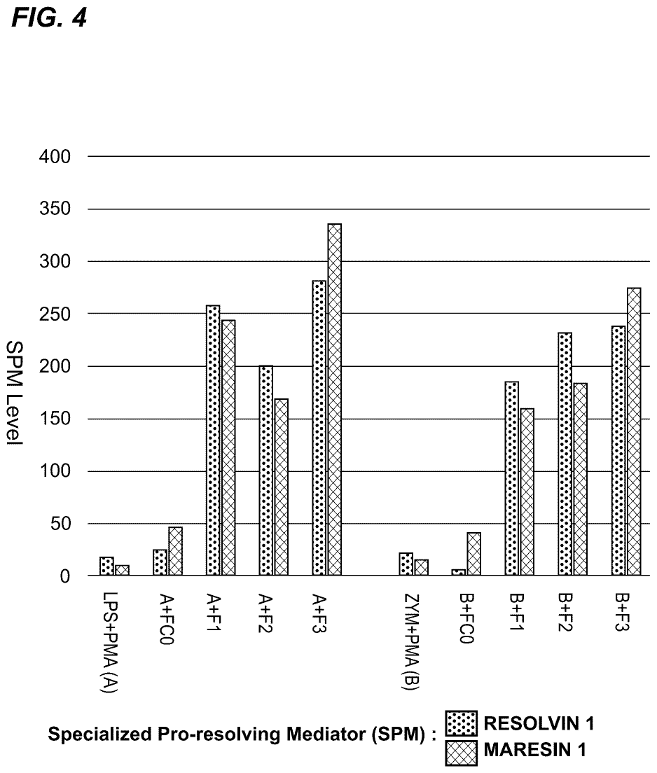

Formulations 1-3, as described in Example 1, were tested in an in vitro cellular periodontal disease model as activators of specialized pro-resolving mediator (SPM) synthesis. The results are shown in FIG. 4.

SPM synthesis is recognized to promote reversal and restorative mode in resolution of acute and chronic inflammation and tissue damage repair and recovery in treating inflammatory diseases. SPMs are specialized pro-resolving anti-inflammatory mediators of inflammation, and they are lipoxins which can be sub-classified into classes such as Resolvins, Maresins, and Protectins. SPMs are generated as a switch to change from inflammatory to anti-inflammatory and pro-regulatory mode for adverse tissue and immune conditions via a unique pathway of cyclooxygenase enzyme inhibition, including the aspirin triggered pathway of COX-2 enzyme inhibition. This example considered the synthesis of two of the prominent members of the SPM family, namely Resolvin D1 (RvD1) and Maresin 1 (MaR1), identified for their pro-regulatory damage-repair and anti-inflammatory properties.

FIG. 4 illustrates the SPM activation properties of Formulations 1-3 using an in vitro cellular periodontal disease model. Cells were cultured from a human patient having end stage periodontitis. The periodontal cells were exposed to pro-inflammatory stimuli PMA in combination with LPS or ZYM (LPS+PMA or ZYM+PMA). Following exposure of the cells to the pro-inflammatory stimuli for 30 minutes, the cells were treated with Formulation C0, Formulation 1, Formulation 2, or Formulation 3. The cells were each exposed to Formulation C0, Formulation 1, Formulation 2, or Formulation 3 for 48 hours, after which the relative SPM synthesis status of Resolvin D1 and Maresin 1 (in combined cellular and released) were determined in all samples. All other conditions were maintained between samples. In FIG. 4, Formulations C0, and Formulations 1-3 are labeled as FC0, F1, F2, and F3.

As shown in FIG. 4, following pro-inflammatory stimulation of the cells, with LPS+PMA or ZYM+PMA, no significant levels of Resolvin D1 and Maresin 1 were synthesized, which signifies a deep inflammatory state. Following treatment of the cells with Formulation C0, the levels of the SPM synthesis remained similar to the inflammatory controls (LPS+PMA or ZYM+PMA), as shown in FIG. 4. Upon treatment of the cells with Formulation 1, Formulation 2, or Formulation 3, each sample exhibited high but comparable levels of the Resolvin D1 and Maresin 1 synthesis, as shown in FIG. 4. This suggests that Formulation 1, Formulation 2, and Formulation 3 are each effective in regulating and reversal of pro-inflammatory disease state to the pro-regulatory therapeutic repair mode.

While not wishing to be bound by theory, it was surprising that even though aspirin mediated COX-2 enzyme inhibition is one of the key factors in the SPM pathway, Formulation C0 was largely ineffective in SPM synthesis. This suggests that aspirin alone may not be sufficient and may require additional catalytic support, such as the presence of cholecalciferol to catalyze the SPM synthesis.

Having described the preferred aspects and implementations of the present disclosure, modifications and equivalents of the disclosed concepts may readily occur to one skilled in the art. However, it is intended that such modifications and equivalents be included within the scope of the claims which are appended hereto.

Also described herein are aspects:

Aspect 1: A composition for maintaining or improving oral health, comprising: cholecalciferol; at least one reversible cyclooxygenase (COX) inhibitor; and at least one irreversible COX inhibitor.

Aspect 2: The composition of aspect 1, wherein the composition comprises from about 0.001 wt. % to about 1.0 wt. %, of the cholecalciferol, based on a total weight of the composition.

Aspect 3: The composition of aspects 1 or 2, wherein the at least one reversible COX inhibitor is selected from the group consisting of ibuprofen, naproxen, diclofenac, indomethacin, ketoprofen, flurbiprofen, piroxicam, meloxicam, sulindac, tolmetin, etodolac, nabumetone, celecoxib, and etoricoxib.

Aspect 4: The composition of any one of aspects 1 to 3, wherein the at least one reversible COX inhibitor is ibuprofen.

Aspect 5: The composition of any one of aspects 1 to 4, wherein the composition comprises from about 0.01 wt. % to about 15.0 wt. %, of the at least one reversible COX inhibitor, based on a total weight of the composition.

Aspect 6: The composition of any one of aspects 1 to 5, wherein the at least one irreversible COX inhibitor is aspirin.

Aspect 7: The composition of any one of aspects 1 to 6, wherein the composition comprises from about 0.001 wt. % to about 2.0 wt. %, of the at least one irreversible COX inhibitor, based on a total weight of the composition.

Aspect 8: The composition of any one of aspects 1 to 7, wherein a ratio by weight of the at least one reversible COX inhibitor to the at least one irreversible COX inhibitor in the composition is from about 10:1 to about 5:1.

Aspect 9: The composition of any one of aspects 1 to 8, further comprising at least one excipient selected from the group consisting of a carrier, a surfactant, a emollient, a thickening agent, a buffering agent, a sweetening agent, a flavoring agent, a coloring agent, an antioxidant, a chelating agent, a preservative, a gelling agent, and combinations thereof.

Aspect 10: The composition of any one of aspects 1 to 9, wherein the composition has a pH of about 6.5 to about 7.5.

Aspect 11: The composition of any one of aspects 1 to 10, wherein the composition is a suspension, a solution, an emulsion, or an emulsified suspension.

Aspect 12: The composition of any one of aspects 1 to 11, wherein the composition is free of ethanol.

Aspect 13: The composition of any one of aspects 1 to 12, wherein the composition is in a form of a gel, a spray, a cream, an ointment, a lotion, a foam, an oral patch, or a liquid.

Aspect 14: The composition of any one of aspects 1 to 12, wherein the composition is combined with a toothpaste or a mouthwash.

Aspect 15: The composition of any one of aspects 1 to 14, further comprising at least one TLR (Toll-like-receptor) inhibitor.

Aspect 16: The composition of any one of aspects 1 to 15, further comprising at least one TLR inhibitor which is a TLR4 inhibitor selected from the group consisting of resatorvid (TAK-242), eritoran, polymyxin B and polymyxin E.

Aspect 17: The composition of any one of aspects 1 to 16, further comprising a TLR inhibitor which is polymyxin B, polymyxin E, or a combination thereof.

Aspect 18: The composition of any one of aspects 1 to 17, wherein the composition comprises from about 0.001 wt. % to about 0.5 wt. %, of the at least one TLR inhibitor, based on a total weight of the composition.

Aspect 19: The composition of any one of aspects 1 to 18, further comprising at least one broad-spectrum antibiotic.

Aspect 20: The composition of any one of aspects 1 to 19, further comprising at least one broad-spectrum antibiotic selected from the group consisting of β-lactams, tetracyclines, fluoroquinolones, sulfonamides, chloramphenicol, macrolides, nitroimidazoles, and combinations thereof.

Aspect 21: The composition of any one of aspects 1 to 19, further comprising at least one broad-spectrum antibiotic selected from the group consisting of amoxicillin, amoxicillin-clavulanate, ampicillin, imipenem, eropenem, doripenem, ertapenem piperacillin, piperacillin-tazobactam, ticarcillin, cephalosporins, carbapenems, monobactams, tetracycline, doxycycline, minocycline, ciprofloxacin, levofloxacin, moxifloxacin, sulfamethoxazole, sulfisoxazole, sulfadiazine, chloramphenicol, and combinations thereof.

Aspect 22: The composition of any one of aspects 1 to 19, further comprising at least one broad-spectrum antibiotic which is amoxicillin or amoxicillin-clavulanate.

Aspect 23: The composition of any one of aspects 1 to 19, further comprising at least one broad-spectrum antibiotic which is a combination of amoxicillin and levofloxacin.

Aspect 24: The composition of any one of aspects 1 to 23, wherein the composition comprises from about 0.01 wt. % to about 10.0 wt. %, of each of the at least one broad-spectrum antibiotic, based on a total weight of the composition.

Aspect 25: A method of maintaining or improving oral health in a subject, comprising: administering an effective amount of the composition of any one of aspects 1-14 to a subject in need thereof, wherein the administering maintains or improves at least one oral health parameter selected from the group consisting of halitosis, tooth sensitivity, gum sensitivity, gum swelling, gum pain and inflammation, gum bleeding, plaque and tarter buildup, and combinations thereof.

Aspect 26: The method of aspect 25, wherein the composition is administered to the subject at least once a day.

Aspect 27: The method of any one of aspects 25 to 26, wherein the composition is administered to the subject at least once a day for greater than about one week.

Aspect 28: The method of any one of aspects 25 to 27, wherein the composition is administered to the subject as part of a daily oral hygiene routine.

Aspect 29: The method of any one of aspects 25 to 27, wherein the composition is administered to the subject as part of a routine cleaning at a dental office.

Aspect 30: The method of any one of aspects 25 to 29, wherein the administering comprises applying the composition for at least about one minute.

Aspect 31: The method of any one of aspects 25 to 30, wherein the administering comprises applying the composition for at least about five minutes.

Aspect 32: The method of any one of aspects 25 to 31, wherein the administering comprises applying the composition topically to at least a portion of an oral cavity of the subject.

Aspect 33: The method of any one of aspects 25 to 32, further comprising removing the composition from the subject.

Aspect 34: The method of any one of aspects 25 to 33, further comprising removing the composition from the subject by having the subject expectorate the composition.

Aspect 35: The method of any one of aspects 25 to 33, further comprising removing the composition from the subject by rinsing the mouth of the subject with a liquid and expectorating the liquid.

Aspect 36: The method of any one of aspects 25 to 35, wherein the composition is substantially not swallowed by the subject.

Aspect 37: The method of any one of aspects 25 to 36, wherein the subject is immunocompromised.

Aspect 38: The method of any one of aspects 25 to 37, wherein the subject is a human.

Aspect 39: The method of any one of aspects 25 to 38, wherein the composition is administered in an amount of about 25 mg to about 150 mg of the total composition per application to the subject.

Aspect 40: A method of preventing or treating periodontal disease in a subject, comprising: administering an effective amount of the composition of any one of aspects 1-24 to a subject in need thereof.

Aspect 41: The method of aspect 40, wherein the administering comprises applying the composition for at least about one minute.

Aspect 42: The method of any one of aspects 40 to 41, wherein the administering comprises applying the composition for at least about five minutes.

Aspect 43: The method of any one of aspects 40 to 42, wherein the administering comprises applying the composition topically to at least a portion of an oral cavity of the subject.

Aspect 44: The method of any one of aspects 40 to 43, wherein the administering comprises applying the composition topically to at least a portion of the oral cavity of the subject which has or is susceptible to the periodontal disease.

Aspect 45: The method of any one of aspects 40 to 44, further comprising removing the composition from the subject.

Aspect 46: The method of any one of aspects 40 to 45, further comprising removing the composition from the subject by having the subject expectorate the composition.

Aspect 47: The method of any one of aspects 40 to 45, further comprising removing the composition from the subject by rinsing the mouth of the subject with a liquid and expectorating the liquid.

Aspect 48: The method of any one of aspects 40 to 47, wherein the composition is substantially not swallowed by the subject.

Aspect 49: The method of any one of aspects 40 to 48, wherein the subject is immunocompromised.

Aspect 50: The method of any one of aspects 40 to 49, wherein the subject is a human.

Aspect 51: The method of any one of aspects 40 to 50, wherein the composition is administered in an amount of about 25 mg to about 150 mg of the total composition per application to the subject.

Aspect 52: The method of any one of aspects 40 to 51, wherein the composition is administered to the subject as part of pre-operative care.

Aspect 53: The method of any one of aspects 40 to 52, wherein the composition is administered to the subject as part of post-operative care.

Aspect 54: The method of any one of aspects 40 to 53, wherein the composition is administered to the subject as part of pre or post-operative care for a procedure selected from the group consisting of scaling and root planning, osseous surgery, root canal, periodontal pocket reduction, flap surgery, soft tissue gum graft, bone graft, LANAP (Laser-Assisted New Attachment Procedure), and dental implant surgery.

Aspect 55: The method of any one of aspects 40 to 54, wherein the periodontal disease is selected from the group consisting of gingivitis, chronic periodontitis, aggressive periodontitis, necrotizing ulcerative gingivitis, necrotizing ulcerative periodontitis, periodontitis as a manifestation of systemic disease, refractory periodontitis, mucogingival defects, and peri-implantitis.

Aspect 56: The method of any one of aspects 40 to 55, wherein the composition is administered to the subject at least once a day for less than one month.

Aspect 57: The method of any one of aspects 40 to 56, wherein the composition is administered to the subject at least once a day for a length of time which is sufficient to treat a bacterial infection.

Aspect 58: The method of aspect 57, wherein following treatment of the bacterial infection, the administering of the composition containing an antibiotic is ceased, and wherein the method further comprises administering an effective amount of the composition of aspects 1-14 to the subject.

What is claimed is:

1. A composition for maintaining or improving oral health, comprising:
about 0.001 wt. % to about 1.0 wt. %, of cholecalciferol, based on a total weight of the composition;
about 0.01 wt. % to about 15.0 wt. %, of at least one reversible COX inhibitor, based on a total weight of the composition; and
about 0.01 wt. % to about 2.0 wt. %, of at least one irreversible COX inhibitor, based on a total weight of the composition.

2. The composition of claim 1, wherein a ratio by weight of the at least one reversible COX inhibitor to the at least one irreversible COX inhibitor in the composition is from about 10:1 to about 5:1.

3. The composition of claim 1, wherein the at least one reversible COX inhibitor is ibuprofen and the at least one irreversible COX inhibitor is aspirin.

4. The composition of claim 1, further comprising at least one excipient selected from the group consisting of a carrier, a surfactant, a emollient, a thickening agent, a buffering agent, a sweetening agent, a flavoring agent, a coloring agent, an antioxidant, a chelating agent, a preservative, a gelling agent, and combinations thereof.

5. The composition of claim 1, wherein the composition has a pH of about 6.5 to about 7.4.

6. The composition of claim 1, wherein the composition is in a form of a suspension, a solution, an emulsion, or an emulsified suspension.

7. The composition of claim 1, wherein the composition is in a form of a gel, a spray, a cream, an ointment, a lotion, a foam, an oral patch, or a liquid.

8. The composition of claim 1, further comprising from about 0.001 wt. % to about 0.5 wt. %, of at least one TLR inhibitor, based on a total weight of the composition.

9. The composition of claim 1, further comprising a TLR inhibitor which is polymyxin B, polymyxin E, or a combination thereof, which are TLR-4 lipopolysaccharide (LPS) binding inhibitors.

10. The composition of claim 8, further comprising about 0.01 wt. % to about 10.0 wt. %, of each of at least one broad-spectrum antibiotic with selectivity for bacterial membrane synthesis and/or DNA synthesis, based on a total weight of the composition.

11. The composition of claim 8, further comprising at least one broad-spectrum antibiotic which is amoxicillin or amoxicillin-clavulanate or is a combination of amoxicillin and levofloxacin.

12. A method of maintaining or improving oral health in a subject, comprising:
administering an effective amount of the composition of claim 1 to a subject in need thereof,
wherein the administering maintains or improves at least one oral health parameter selected from the group consisting of halitosis, tooth sensitivity, gum sensitivity, gum swelling, gum pain and inflammation, gum bleeding, plaque and tarter buildup, and combinations thereof.

13. The method of claim 12, wherein the composition is administered to the subject at least once a day wherein each administration is for at least about one minute.

14. The method of claim 12, wherein the administering comprises applying the composition topically to at least a portion of an oral cavity of the subject.

15. The method of claim 12, wherein the composition is administered in an amount of about 25 mg to about 150 mg of the total composition per application to the subject.

16. A method of preventing or treating periodontal disease in a subject, comprising:
administering an effective amount of the composition of claim 10 to a subject in need thereof.

17. The method of claim 16, wherein the administering comprises applying the composition topically to at least a portion of the oral cavity of the subject which has or is susceptible to the periodontal disease.

18. The method of claim 16, wherein the periodontal disease is selected from the group consisting of gingivitis, chronic periodontitis, aggressive periodontitis, necrotizing ulcerative gingivitis, necrotizing ulcerative periodontitis, periodontitis as a manifestation of systemic disease, refractory periodontitis, mucogingival defects, and peri-implantitis.

19. The method of claim 16, wherein the composition is administered to the subject at least once a day for a length of time which is sufficient to treat a bacterial infection.

20. The method of claim 19, wherein following treatment of the bacterial infection, the administering of the composition containing an antibiotic is ceased, and wherein the method further comprises administering an effective amount of a composition comprising:
about 0.001 wt. % to about 0.5 wt. %, of cholecalciferol, based on a total weight of the composition;
about 0.01 wt. % to about 15.0 wt. %, of at least one reversible COX inhibitor, based on a total weight of the composition; and
about 0.001 wt. % to about 2.0 wt. %, of at least one irreversible COX inhibitor, based on a total weight of the composition.
